(19) United States
(12) Patent Application Publication
(10) Pub. No.: US 2025/0288603 A1
(43) Pub. Date: Sep. 18, 2025

(54) ANIMAL FOOD COMPOSITION COMPRISING A SOURCE OF GLYCYRRHIZIN

(71) Applicant: MARS, INCORPORATED, McLean, VA (US)

(72) Inventor: Adrian WATSON, Aimargues (FR)

(73) Assignee: MARS, INCORPORATED, McLean, VA (US)

(21) Appl. No.: 18/859,858

(22) PCT Filed: Apr. 27, 2023
(86) PCT No.: PCT/US2023/020259

§ 371 Date: Oct. 24, 2024

(30) Foreign Application Priority Data
Apr. 27, 2022 (EP) 22170352.3

(51) Int. Cl. A61K 31/704; A23K 20/121; A23K 20/174; A23K 50/40; A61K 36/484

(52) U.S. Cl. CPC A61K 31/704; A23K 20/121; A23K 20/174; A23K 50/40; A61K 36/484

(57) ABSTRACT

The present disclosure relates to an animal food composition comprising a source of glycyrrhizin for use for preventing and/or treating allergic inflammatory skin diseases wherein the said animal food composition is formulated to provide an animal a daily amount of glycyrrhizin in an amount of at least about 0.02 mg/kg of bodyweight.

### 1. CROSS-REFERENCE TO RELATED APPLICATIONS

This application claims the benefit of priority to European Patent Application Serial No. 22170352.3 filed on Apr. 27, 2022, the contents of which are incorporated herein by reference in their entirety.

### 2. FIELD OF THE DISCLOSURE

The presently disclosed subject matter relates to the field of animal food compositions for preventing and/or treating allergic inflammatory skin diseases, in particular for preventing and/or treating atopic dermatitis.

### 3. BACKGROUND OF THE DISCLOSURE

Reports strongly suggest that the prevalence of allergic diseases is increasing, particularly in Western societies (Pawankar et al., World Allergy Organization. (2013) 11-9).

Allergic inflammatory skin diseases are chronic disorders characterized by clinical signs such as erythema, pain, redness, swelling, and the appearance of small vesicles or papules on the skin. They are the second most common allergic skin disease in animals, in particular dogs, surpassed only by flea allergies. These allergic reactions can be brought on by normally harmless substances such as grass, mold spores, house dust mites, and other environmental allergens.

The primary symptoms are skin inflammation and pruritus usually associated with scratching and excoriation by the affected animal caused by an interaction between genetic and environmental factors. The allergic symptoms appear as eczematous skin, and animals (such as dogs) with atopic dermatitis often suffer from pruritus, or severe itching, hair loss, excoriation of the skin from deep scratching, frequent licking of their paws and excessive tear production. Secondary skin problems are also common, including skin infections and excessive sebum discharge. Particularly, atopic dermatitis is one of the most common allergic inflammatory skin diseases.

A common feature of such diseases across multiple manifestations is an immune response bias towards expression of so-called T-helper 2 cell (Th-2) cytokines, the expression of inflammatory mediators which are associated with promotion of IgE and eosinophilic responses. For example, increased Interleukin IL-4 or IL-5, relative to Th-1 helper cell cytokines such as Interferon-γ (IFN-7) or Th-17 helper cell cytokines such as IL-17, seems to predispose the immune response towards the allergic phenotype. Th-1 cytokine IFN-7, on the other hand, is known to inhibit IgE synthesis, Th-2 cell proliferation, and IL-4 receptor expression on T cells (Spergel et al., Am J Clin Derm. 9 (2008) 233-234, Laurence et al., Natural Immunology. 8 (2007) 903-905, and Okamoto et al., Clinical and Experimental Immunology. 157 (2009) 27-34). As such, bioactives that modulate the immune response to suppress Th-2, and even promote Th-1 cell proliferation and cytokine production, can prove useful in the management of allergies or allergic disease.

Several treatments for atopic dermatitis and pruritus have already been described including the use of ultra-micronized palmitoylethanolamide (Noli et al.; Vet Dermatol 2015; 26, 432-40) or antihistamines, such as fexofenadine, or drugs such as cyclosporine. Nevertheless, most of the available medications are not suitable for long term use due to significant side-effects (as well as, in many cases, prohibitive cost). Although immunotherapy can improve signs if the allergens are identified, success rates remain variable. The ability to reduce the dependency on medications would be seen as a valuable contribution to the field and is the primary aim of nutritional interventions.

Indeed, there is evidence now that genetic predisposition can influence the immune system as well as functional/structural aspects of the skin barrier. Known environmental components are allergen load and the microbial population present on the skin. Drugs currently prescribed for allergic conditions target individual aspects of the disease ‘march’, typically immune related, but only control the disease rather than offering long term relief or offer a cure. A nutritional management strategy is more likely to be successful if it targets multiple aspects simultaneously.

Numerous natural products derived from plant sources have shown the potential for immune modulation (Block et al., Integrative Cancer Therapies. 2 (2003) 247-267, Gertsch et al., Journal of Ethnopharmacology. 136 (2011) 385-391 and Licciardi et al., Int Immunopharmacol. 11 (2011) 390-398). Some of these phytochemical products are reputed to modulate immunological pathways relevant to allergic conditions. However, the level of scientific support for these beneficial activities is variable and, in some cases, there is little support beyond recorded traditional use.

In particular, focus has been placed on Glycyrrhiza glabra licorice root which has previously shown immunomodulatory properties. Indeed, studies have shown a prominent chemical constituent of licorice, glycyrrhizin, also known as glycyrrhizic acid, affects maturation and signalling activities of dendritic cells (Bordbar et al., Immunopharmacol Immunotoxicol. 36 (2014) 52-60, Kim et al., Immunopharmacol Immunotoxicol. 35 (2013) 329-35 and Aipire et al., Sci Rep. 7 (2017) 43796) with suppression of certain Th-2 type responses and a concomitant tendency towards Th-1 responses (Bordbar et al., Cell Immunol. 280 (2012) 44-49 and Hua et al., Int Immunopharmacol. 12 (2012) 518-25). In addition to glycyrrhizin, other significant components of licorice, namely isoliquiritigenin and naringenin, have been shown to influence T-regulatory cell induction and function (Guo et al., Sci Rep. 5 (2015) 14046).

As such, glycyrrhizin has been included in animal food compositions in the past in order to prevent and/or treat allergic inflammatory diseases: For example, WO2021/003280 describes the use of (i) a source of glycyrrhizin in combination with (ii) a source of one or more curcuminoids in an animal for composition for this purpose, the disclosure of which is incorporated herein in its entirety.

However, the amount of glycyrrhizin that can be included in animal food composition is usually very low. Indeed, it has been previously shown that an association exists between glycyrrhizin consumption and hypertension in dogs. Therefore, the official safety advice for glycyrrhizin is that the Presumed Safe Intake (PSI), in particular for dogs, is 1 mg/kg of complete feed, providing an estimated 0.3 mg/day or 0.02 mg/kg of bodyweight (Bwt) per day (Bwt/day) (EFSA Journal, 2015 recommendations based on the study by van Gelderen et al., Glycyrrhizic acid: the assessment of a no effect level, Human & Experimental Toxicology, 2000, 19, 434-439).

As such, the amount of active compound included in the compositions, and as such that can provide the anti-inflammatory properties which are sought, is limited. Thus, there remains a need for treatment options for allergic inflammatory skin diseases, such as atopic dermatitis. There also remains a need for alternative natural products for treating or preventing such diseases. Thus, there remains a need for novel diets designed to improve both immune function and barrier defences.

Further, there remains a need for novel treatment options, in particular novel diets, for allergic inflammatory skin diseases, such as atopic dermatitis, which is particularly efficient on the disease but is also safe and presents limited side-effects for the individual to be treated.

### 4. SUMMARY

The purpose and advantages of the disclosed subject matter will be set forth in and are apparent from the description that follows, as well as will be learned by practice of the disclosed subject matter.

A first aspect of the present disclosure relates to an animal food composition comprising a source of glycyrrhizin for use in a method for preventing and/or treating inflammatory diseases or disorders, allergic inflammatory skin diseases, wherein the said animal food composition is formulated for providing to an animal a daily amount of glycyrrhizin in an amount of at least about 0.02 mg/kg of bodyweight.

In particular, the animal food composition can be formulated for providing to an animal a daily amount of glycyrrhizin in an amount of from about 0.02 mg/kg of bodyweight to about 2 mg/kg of bodyweight.

In particular, the animal food composition can be formulated for providing to an animal a daily amount of glycyrrhizin in an amount of from about 0.03 mg/kg to about 2 mg/kg of bodyweight, in particular from about 0.05 mg/kg to about 1 mg/kg of bodyweight, in particular from about 0.1 mg/kg to about 0.8 mg/kg of bodyweight, in particular from about 0.2 mg/kg to about 0.6 mg/kg of bodyweight.

The present disclosure also relates to an animal food composition comprising a source of glycyrrhizin for use as a medicament, wherein the said animal food composition is formulated for providing to an animal a daily amount of glycyrrhizin in an amount of at least about 0.02 mg/kg of bodyweight.

In a particular embodiment, the animal food composition is selected from a group consisting of a nutritionally complete animal food composition, a food supplement, and a topper composition In a particular embodiment, the source of glycyrrhizin of the animal food composition comprises a licorice root extract.

In a particular embodiment, the animal food composition further comprises linoleic acid.

In a particular embodiment, the animal food composition further comprises EPA/DHA, Taurine, Lutein, Vitamin E and/or a combination thereof.

In a particular embodiment, the allergic inflammatory skin disease is selected from the group consisting of atopic dermatitis, flea allergic dermatitis, urticaria, insect bite allergy, angioedema, inhalant allergy, inhalant allergic dermatitis, food allergic dermatitis, contact dermatitis, miliary dermatitis, eosinophilic granuloma, head and neck pruritus, and generalized pruritus.

A second aspect of the present disclosure relates to a kit comprising:
(i) a first part comprising a source of glycyrrhizin; and
(ii) a second part comprising one or more additional ingredients;
for use in a method for preventing and/or treating inflammatory diseases or disorders, allergic inflammatory skin diseases, wherein the said kit is formulated for providing to an animal a daily amount of glycyrrhizin in an amount of at least about 0.02 mg/kg of bodyweight.

A third aspect of the present disclosure relates to an animal food composition comprising a source of glycyrrhizin, wherein the amount of glycyrrhizin is at least about 5 mg/kg based on the total weight of dry matter of the animal food composition.

In particular, the amount of glycyrrhizin can be from about 5 mg/kg to about 100 mg/kg based on the total weight of dry matter of the animal food composition.

In particular, the amount of glycyrrhizin can be from about 5 mg/kg to 100 mg/kg, in particular from about 10 mg/kg to about 75 mg/kg, in particular from about 15 mg/kg to about 50 mg/kg and in particular from about 20 mg/kg to 45 mg/kg based on the total weight of dry matter of the animal food composition.

In a particular embodiment, the animal food composition comprises a kibble and/or the animal food composition further comprises a source of proteins.

In particular, the animal food composition comprises a nutritionally complete food composition.

In particular, the animal food composition or kit is formulated for oral administration.

In particular, the source of glycyrrhizin of the animal food composition or in the kit comprises a licorice root extract.

In particular, the animal food composition may comprise one or more additional ingredients.

In particular, the one or more additional ingredients of the animal food composition or of the kit can be selected from linoleic acid, EPA/DHA, Taurine, Lutein, Vitamin E and/or a combination thereof.

The foregoing has outlined broadly the features and technical advantages of the present application in order that the detailed description that follows may be better understood.

Additional features and advantages of the application will be described hereinafter which form the subject of the claims of the application. It should be appreciated by those skilled in the art that the conception and specific embodiment disclosed may be readily utilized as a basis for modifying or designing other structures for carrying out the same purposes of the present application. It should also be realized by those skilled in the art that such equivalent constructions do not depart from the spirit and scope of the application as set forth in the appended claims. The novel features which are believed to be characteristic of the application, both as to its organization and method of operation, together with further objects and advantages will be better understood from the following description.

### 5. BRIEF DESCRIPTION OF THE DRAWINGS

FIG. 1 shows the survival of Jurkat cells and human peripheral blood mononuclear cells (PBMCs) in the presence of increasing concentrations of licorice root extract (LRE). Cell viability was assessed by trypan blue exclusion. Data for both assays are expressed as the cell survival (empty diamond “⋄”: viability of PBMCs; full circle “●”: viability of Jurkat cells) relative to a +ve control with anti-CD3/CD28 stimulation alone (Mean+/−SEM). * denotes statistically significant reduction (P<0.01) in cell viability. From these assays a LRE concentration of 100 mg/ml was chosen as the cut-off point for interpretation of cytokine assays. * denotes LRE concentrations at which cell viability was reduced for both Jurkat and PBMC populations (p<0.01).

FIG. 2 represents Human T-cell (PBMC) proliferation and interleukin-2 (IL-2) release. Cell proliferation (full circle “●”: mean cell proliferation) was induced via a combination of anti-CD3/CD28 stimulation and assessed by the incorporation of [3H]thymidine into proliferating T-cells. Interleukin-2 release (empty diamond “⋄”: mean IL-2 release): from activated cells was measured by ELISA and is shown on the same axis. In both cases data are expressed as activity relative to activation alone +ve control (Mean+/−SEM). The licorice extract showed little effect on T-cell proliferation or IL-2 release at concentrations previously shown to be non-cytotoxic (<100 mg/ml). At 100 mg/ml there was evidence of an effect on cell proliferation. * denotes LRE concentration at which IL-2 expression was significantly reduced (p<0.05); # denotes LRE concentration at which cell proliferation was significantly reduced (p<0.05).

FIG. 3 shows the effect of licorice root extract on the release of IFN-γ (empty square “□”), interleukin-17 (empty circle “◯”), and interleukin-5 (empty triangle “Δ”), by anti-CD3/CD28 activated human T-cells. Data are expressed as concentration of cytokine relative to an activation alone +ve control (Mean+/−SEM). * denotes licorice extract concentrations that significantly inhibit IL-5 release alone; # denotes licorice extract concentrations that significantly inhibit release of all 3 cytokines (p<0.05).

### 6. DETAILED DESCRIPTION

The presently disclosed subject matter is directed to making available a product for preventing and/or treating inflammatory diseases or disorders, allergic inflammatory skin diseases, especially atopic dermatitis, in animals, especially in dogs, i.e., canine atopic dermatitis. In particular, this product can be useful to support the management of canine atopic dermatitis.

The presently disclosed subject matter provides herein an animal food composition comprising a source of glycyrrhizin in an amount that is higher than the recommended daily dose.

Surprisingly, the presently disclosed subject matter has determined that an aqueous extract of licorice root can inhibit release of Interleukin-5 (IL-5) from activated T-cells within a concentration range which is non-cytotoxic and does not stimulate proliferation nor Interleukin-2 (IL-2) release. Further, the presently disclosed subject matter was able to demonstrate that the consumption of an amount of glycyrrhizin that is more than about 20 times higher than the recommended daily dose for dogs did not induce any significant blood pressure homeostasis in adult dogs. Still further, the presently disclosed subject matter has shown that the consumption of an amount of glycyrrhizin that is more than about 20 times higher than the recommended daily dose for dogs did not induce any change in the potassium excretion nor in aldosterone secretion, which results denote that this high dose of glycyrrhizin does not affect kidney function.

Hence, the presently disclosed subject matter relates to an animal food composition comprising a source of glycyrrhizin allowing its provision to animals, especially canine, such as dogs, of an amount higher than the usual recommended amount, while such a food composition remains safe, i.e., does not cause an increased blood pressure and does affect kidney function, such as does not cause an increased potassium excretion nor aldosterone secretion. Such a composition is useful for preventing and/or treating inflammatory diseases or disorders, allergic inflammatory skin diseases in animal, and more particularly an animal affected with atopic dermatitis. In particular embodiments, the animal is a canine. In certain embodiments, the canine is a dog.

As used herein, inflammatory diseases or disorders encompass inflammation of the gastrointestinal tract.

Further, the presently disclosed subject matter provides an animal food composition which presented good overall palatability and digestibility, in particular in dogs, and remained stable throughout its manufacturing process.

More particularly, the presently disclosed subject matter also provides an animal food composition, which food composition can encompass a food supplement, a topper composition and/or a complete food composition. The animal food composition disclosed herein, either consisting of a food supplement or of a complete food composition, comprises a source of glycyrrhizin in amounts specified herein. In some embodiments, the food composition is in the form of a wet food composition, such as a gravy composition. Alternatively, the food composition is in the form of a dry food composition, such as in the form of a kibble.

For clarity, but not by way of limitation, the detailed description of the presently disclosed subject matter is divided into the following subsections:
6.1. Definitions;
6.2. Animal Food Compositions;
6.3. Methods of Preparing Animal Food Compositions; and
6.4. Therapeutic Applications.

### 6.1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the present disclosure and how to make and use them.

For purposes of interpreting this specification, the following definitions will apply and whenever appropriate, terms used in the singular will also include the plural and vice versa. In the event that any definition set forth below conflicts with any document incorporated herein by reference, the definition set forth below shall control.

As used herein, the use of the word “a” or “an” when used in conjunction with the term “comprising” in the claims and/or the specification can mean “one,” but it is also consistent with the meaning of “one or more,” “at least one,” and “one or more than one.”

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to systems or processes, the term can mean within an order of magnitude, preferably within 5-fold, and more preferably within 2-fold, of a value.

The terms “comprise(s),” “include(s),” “having,” “has,” “can,” “contain(s),” and variants thereof, as used herein, are intended to be open-ended transitional phrases, terms or words that do not preclude additional acts or structures. The present disclosure also contemplates other embodiments “comprising,” “consisting of” and “consisting essentially of,” the embodiments or elements presented herein, whether explicitly set forth or not.

As used herein, the term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value.

As used herein, the term “allergic” refers to a deregulated or unregulated sensitivity, in particular a hypersensitivity, of the immune system to typically harmless substances, such as those which are present in the environment. As previously mentioned herein, allergic inflammatory skin disease is characterized by producing erythema, pain, redness, swelling, and small vesicles or papules on the skin. Body regions that are frequently affected in atopic dermatitis include the head, pinnae, feet, ventral abdomen and axillae. Common allergy triggers include but are not limited to: airborne allergens, such as pollen, animal dander, dust mites and mold; certain foods, particularly peanuts, tree nuts, wheat, soy, fish, shellfish, eggs and milk; insect stings, such as from a bee or wasp; medications, particularly penicillin or penicillin-based antibiotics; latex or other substances you touch, which can cause allergic skin reactions; allergic inflammatory skin disease encompasses atopic dermatitis, flea allergic dermatitis, urticaria, insect bite allergy, angioedema, inhalant allergy, inhalant allergic dermatitis, food allergic dermatitis, contact dermatitis, miliary dermatitis, eosinophilic granuloma, head and neck pruritus and generalized pruritus. It will thus be understood herein that the term “allergic” can relate both to “auto-antigens” and to exogenous antigens.

As used herein, the term “animal” or “animals” designates a ruminant, poultry, swine, mammal, horse, mouse, rat, rabbit, guinea pig, hamster, cow, cat, or canine. In specific embodiments, the term animal is directed to a canine. For example, an animal can be, without limitation, a penguin, falcon, agouti, american kestrel, snake, bear, condor, ant, impala, antelope, armadillo, australian brush-turkey, babirusa, bald eagle, bali myna, fox, bat, sunbird, bee, beetle, bear cat, binturong, bird of paradise, boa, bonobo, bontebok, brown bear, caecilian, camel, caracal, chameleon, cheetah, chimpanzee, chinchilla, cichlid, clouded leopard, cobra, cockatoo, crocodilian, eagle, dhole, dolphin, platypus, echidna, elephant, emu, flamingo, fossa, frog, toad, galapagos, gelada, giant anteater, giant panda, gila monster, giraffe, goat, sheep, frog, gorilla, gouldian finch, guam rail, guanaco, guenon, hippo, hornbill, ibis, iguana, jacana, jaguar, kagu, kangaroo, wallaby, kingfisher, kingsnake, kinkajou, kiwi, klipspringer, koala, koi, komodo dragon, ladybug, laughing kookaburra, lemur, leopard, lion, lizard, lynx, macaw, magpie goose, manatee, mandrill, mangabey, marsupial, meerkat, metallic starling, monkey, ocelot, okapi, opossum, orangutan, oryx, ostrich, otter, owl, painted dog, parrot, peafowl, pelican, polar bear, porcupine, prairie dog, pronghorn, przewalski's horse, raccoon, radjah shelduck, red panda, reindeer, rhinoceros, ringtail, rock hyrax, saiga, scorpion, sea lion, secretary bird, serval, siamang, snow leopard, spider, stork, sun bear, surinam toad, takin, tapir, tasmanian devil, tenrec, tiger, toucan, tuatara, turtle, vulture, warthog, waxbill, whale, cape buffalo, wolf, wombat or zebra.

As used herein, the term “canine” encompasses animals, including pet animals, selected in the group comprising wolf, coyote, jackal, dingo, dhole, fox, raccoon dog, and dogs. As used herein, “dogs” encompass wild dogs and domestic dogs. In specific embodiments, the term dogs means domestic dogs.

As used herein, the term “drug” designates any substance or composition represented as having treatment, curative or prevention properties against animal diseases. By extension, a medicinal product includes any substance or composition that can be used or administered to animals for the purpose of making a medical diagnosis or restoring, correcting or modifying their physiological functions by exercising a pharmacological, immunological or metabolic action.

In the detailed description herein, references to “embodiment,” “an embodiment,” “one embodiment,” “in various embodiments,” etc., indicate that the embodiment(s) described can include a particular feature, structure, or characteristic, but every embodiment might not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

As used herein, the term “enantiomers” refers to a pair of stereoisomers that are non-superimposable mirror images of each other. A 1:1 mixture of a pair of enantiomers is a “racemic” mixture or a racemate. The term is used to designate a racemic mixture where appropriate.

As used herein, the term “enantiopure” refers to a sample that within the limits of detection consists of a single enantiomer.

As used herein, the term “diastereoisomers” refers to stereoisomers that have at least two asymmetric atoms, but which are not mirror-images of each other. The absolute stereochemistry is specified according to the Cahn-Ingold-Prelog R S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line. The compounds of the presently disclosed subject matter contain one or more asymmetric centers and can thus give rise to enantiomers, diastereomers, and other stereoisomeric forms that can be defined, in terms of absolute stereochemistry, as (R)- or (S)-. The presently disclosed subject matter is meant to include all such possible isomers, including racemic mixtures, optically pure forms, and intermediate mixtures. Optically active (R)- and (S)-isomers can be prepared using chiral synthons or chiral reagents or resolved using conventional techniques. If the compound contains a double bond, the substituent can be E or Z configuration. If the compound contains a disubstituted cycloalkyl, the cycloalkyl substituent can have a cis- or trans-configuration. All tautomeric forms are also intended to be included.

As used herein, the term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Therefore, the term stereoisomer includes enantiomers, diastereomers, or racemates of the compound. Also, as used herein, the terms “constitutional isomers” refers to different compounds which have the same numbers of, and types of, atoms but the atoms are connected differently, such as, but not limited to tautomers.

As used herein, the term “food composition” or “diet” covers all of foodstuff, diet, food supplement, or a material that can contain proteins, carbohydrates and/or crude fats. Foods can also contain supplementary substances or additives, for example, minerals, vitamins and condiments (See Merriam-Webster's Collegiate Dictionary, 10th Edition, 1993). Such food compositions can be nutritionally complete or not. In one embodiment, an animal food composition according to the presently disclosed subject matter is a nutritionally complete food composition. Illustratively, a food composition as described herein can include, but it is not limited to, protein, crude fat, ash, crude fiber, starch, calcium, phosphorus, sodium, chloride, potassium, magnesium, iron, water, copper, manganese, zinc, selenium, vitamin A, vitamin D3, vitamin B1, vitamin B2, vitamin B6, vitamin B12, vitamin B7, vitamin B9, cholin chloride, arachidonic acid, W3 fatty acid or W6 fatty acid.

As used herein, a “food supplement” designates a concentrated source of nutrients, substances for nutritional or physiological purposes, or plants and plant preparations intended to compensate for deficiencies in an animal's regular diet. A food supplement is usually in the form of capsules, lozenges, tablets, pills, powder packets, or liquid forms (ampoules, vials with droppers).

As used herein, the term “inflammation” refers to a local response to cellular injury that is marked by capillary dilatation, leukocytic infiltration, redness, heat, and pain.

As used herein, “nutritionally complete” means that the composition provides the complete and balanced nutritional requirement to animals, and in particular embodiments to dogs. Therefore, a nutritionally adequate feed is a feed with which the said animal, e.g., the said dog, can be fed as the sole ration and is capable of sustaining life without additional food (except water). The food composition can contain a carrier, a diluent, or an excipient. Depending on the intended use, the carrier, diluent, or excipient can be chosen to be suitable for animal use, e.g., for canine use such as dogs. In a general manner, nutritionally complete compositions comprise at least one source of proteins (or polypeptides), such as protein extracts, at least one source of, vitamins, minerals, trace elements, and fats.

As used herein, the term “preventing”, can include the reduction of a likelihood of occurrence, or of re-occurrence, of a given condition in an animal.

As used herein, the term “treating” or “treatment” as used herein denotes reversing, alleviating, inhibiting the progress of, or improving the disorder, disease or condition to which such term applies, or one or more symptoms of such disorder, disease or condition

As used herein, the term “wet food” or “wet food composition” generally refers to a food composition having a moisture content of about 30% or more, generally of more than about 40% by weight, relative to the total weight of the food composition. Preferably, the wet food composition has a moisture content lower than about 90% in weight, relative to the total weight of the food composition. In general, it is the final product of a process comprising a final step of sterilization (instead of a drying step). In particular embodiments, the wet food comprises a chunk form, more particularly of chunks in gravy form. Preferably, the wet food comprises chunks and gravy, chunks in jelly, loaf, mousse, terrine, bites form. “Chunks and gravy” products comprise a preformed meat particle prepared by making a meat emulsion and by putting this meat emulsion through a muzzle under pressure and then cooked. A product, such as cooked meat, is diced into chunks, which are eventually mixed with a gravy or sauce. The two components are then filled into a container, usually a can or pouch, which is seamed or sealed and sterilized. As opposed to the ground loaf, chunk and gravy compositions have physically separated, discrete chunks (i.e., pieces of ground meat and grains) as prepared. These discrete particles are present in the gravy-type liquid in the final container. When serving, chunk and gravy products flow out of the can and can be easily mixed with other dry products. While the chunk and gravy products allow better integrity of the individual ingredients, the heterogeneous formulation of the chunk and gravy products are sometimes disfavored by consumers. Wet food compositions are generally packaged in can-like containers and are considered “wet” in appearance because of the moisture contained therein. Two types of wet compositions are generally known in the art. The first is known in the art as “ground loaf.” Loaf products are typically prepared by contacting a mixture of components under heat to produce an essentially homogeneous, intracellular honeycomb-type mass or “ground loaf.” The ground loaf mass is then packaged into a cylindrical container, such as a can. Upon packing, ground loaf assumes the shape of the container such that the ground loaf must be cut when serving to a companion animal. The wet food composition is preferably packaged. In this way, the consumer is able to identify, from the packaging, the ingredients in the food product and confirm that it is suitable for the particular pet in question. The packaging can be metal, plastic, paper or card.

As used herein, the term “dry food” or “dry food composition” generally refers to a food or composition having a moisture content of less than about 12% by weight, relative to the total weight of the food composition, and commonly even less than about 7% by weight, relative to the total weight of the food composition. In particular embodiments, dry food according to the presently disclosed subject matter has a moisture content of at most about 12% by weight. In some embodiments, the said dry food has a moisture content of about 7% or less, such as about 5% by weight. In particular embodiments, the dry food has a moisture content of more than about 3% by weight, relative to the total weight of the food composition. For instance, the examples provided herein illustrate a dry food having a moisture content of about 9.5% by weight, relative to the total weight of the food composition. In certain embodiments, the dry food consists of a kibble. In non-limiting examples, kibbles include particulates; pellets; pieces of pet food, dehydrated meat, meat analog, vegetables, and combinations thereof; and pet snacks, such as meat or vegetable jerky, rawhide, and biscuits. The dry food composition can be manufactured by mixing together ingredients and kneading in order to make consistent dough that can be cooked. In general, it can be the final product of a process comprising an extrusion step followed by a drying step.

The process of creating a dry food is usually done by baking and/or extruding. The dough is typically fed into a machine called an expander and/or extruder, which uses pressurized steam or hot water to cook the ingredients. While inside the extruder, the dough is under extreme pressure and high temperatures. The dough is then pushed through a die (specifically sized and shaped hole) and then cut off using a knife. The puffed dough pieces are made into kibble by passing it through a dryer so that moisture is dropped down to a defined target ensuring stability of the food until consumption. The kibble can then be sprayed with fats, oils, minerals, vitamins, the natural extracts cocktail and optionally sealed into packages. The dry food composition is preferably packaged. In this way, the consumer is able to identify, from the packaging, the ingredients in the food product and confirm that it is suitable for the particular pet, i.e., dog, in question. The packaging can be metal, plastic, paper or card.

As used herein, the term “semi-moist food” or “semi-moist food composition” generally refers to a food composition with an intermediate moisture content of about 12% to about 30% in weight, relative to the total weight of the food composition. Hence, such semi-moist food composition is generally the final product of a process allowing a moisture content value that is intermediate between a dry food and a wet food. In some embodiments, the said process can comprise a step of adding a humectant agent. In some embodiments, the said process comprises an extrusion step and a subsequent treatment step with Super-Heated Stream (SHS). In some embodiments, the semi-moist food according to the presently disclosed subject matter containing more than about 12% and at most about 30% moisture by weight, relative to the total weight of the food composition. Illustratively, a semi-moist food composition has about 11% to about 20% moisture by weight, relative to the total weight of the food composition, and/or a water activity of about 0.64 to about 0.75, preferably both.

As non-limitative example, semi-moist food can be obtained using Super-Heated Stream (SHS) processes such as processes or methods described in the published patent applications WO2009/018990, WO2009/018996, WO2010/112097, WO2014/122072, WO2016/071372 and/or WO2016/071367, the disclosures of which are incorporated herein by reference in their entireties.

As disclosed herein, in certain embodiments, when the animal food composition is a semi-moist food, it consists of soft semi-moist kibbles.

As disclosed herein, “moisture content” of an animal food composition can be determined by a Loss on Drying Method, comprising the steps of:
(a) Weighing a sample of a food composition, thereby obtained the total weight;
(b) Heating, such as 135±2° C. for 240 minutes, the sample of step a) in an oven until the moisture is all driven off, thereby obtained a dry sample;
(c) Weighing the dry sample; thereby obtained the dry weight;
(d) Calculating the difference between the total weight and the dry weight, thereby obtaining the moisture content, as:

$$Moisture Content = ( total weight ) - ( dry weight )$$

As used herein, the term “water activity” refers to the ratio of the partial vapor pressure of water in a food composition divided by the standard state partial vapor pressure of water. Several methods, known to the skilled person, can be employed to measure the water activity including a resistive electrolytic, a capacitance or a dew point hygrometer.

Most preferably, it is referred to as the method prescribed by the International Standard ISO 21087 relating to determining water activity in animal food and animal feeding stuffs.

As used herein a “solvent” can refer to acetic acid, n-butanol, isopropanol, n-propanol, ethanol, methanol, formic acid, dimethylformamide, dimethyl sulfoxide, acetonitrile, acetone, dichloromethane, tetrahydrofuran, ethyl acetate, n-hexane, benzene, toluene, diethyl ether, chloroform, 1,4-dioxane, water or a combination thereof, preferably to water. In non-limiting embodiments, the solvent is a mixture of about 80% w/w of water and about 20% w/w of ethanol.

As used herein, the term “weight percent” or “wt %” or “by weight” is meant to refer to either (i) the quantity by weight of a constituent/component in a composition as a percentage of the total weight of the composition; or (ii) the quantity by weight of a constituent/component in the material as a percentage of the weight of the final material or product. All weight percentages expressed herein are by weight of the total weight of dry matter of the food composition unless expressed otherwise.

As used herein, an amount of a component as expressed as weight/Mcal consists of a weight amount of the said component by unit of Metabolizable Energy (ME) of the total animal food composition. As used herein, the ME parameter is intended to represent the energy value of a food composition that is directly metabolized after consumption. Within the scope of the presently disclosed subject matter, the ME value can be measured by any suitable method known in the art. Illustratively, the ME value can be measured using feeding trial. In practice, the gross energy (GE) of the food is determined in the laboratory, and the amounts of food eaten by the animals are recorded. The feces and urine from the animals are collected, and the energy in each is determined and called fecal energy (FE) and urinary energy (UE), respectively. The ME is then calculated as follows:

$$M E ( kcal / kg ) = [ G E - ( F E + U E ) ] / Kg of food consumed .$$

Alternatively, the ME value can be measured by a mathematical method, in particular taking into account the percentage of crude fat (CF), of crude protein (CP), and NFE (carbohydrates) in the composition. In practice each percentage is multiplied by its respective Atwater Factors. The resulting sum is then multiplied by 10. The mathematical method can be represented by the following formula:

$$M E ( kcal / kg ) = 10 \times [ ( 35 \times C P ) + ( 85 \times C F ) + ( 35 \times NFE ) ] .$$

Metabolizable Energy is conventionally determinable according to standard methods, and especially according to the National Research Council.

As used herein, an amount of a component as expressed as weight/kg of food composition, e.g., weight/kg of complete food, consists of a weight amount of the said component by unit of weight, in kilograms, of the food composition, e.g., of the said complete food. As used herein, “complete food” can refer to the quantitative mixture of all dietary ingredients that have been formulated to meet the energy, protein, fat, vitamin and mineral requirements of an animal. As used herein, an amount of component as expressed as weight/kg of body weight (Bwt)/day is the daily amount of the said component by unit of the bodyweight, in kilograms, of an animal, e.g., of a dog. In certain embodiments, the weight/kg of bodyweight refers to the weight of the component fed to the animal by kg of bodyweight of said animal.

### 6.2. Animal Food Compositions

In certain embodiments, the animal food composition for preventing and/or treating inflammatory diseases or disorders, can comprise glycyrrhizin, additional ingredients, and additives. In particular embodiments, the additional ingredients can include curcuminoids, linoleic acid, EPA/DHA, taurine, lutein, vitamin E, or combinations thereof. In certain embodiments, the additives can include protein, crude fat, fibers, nitrogen free extract, carbohydrates, starch, and/or combinations thereof.

In certain embodiments, the presently disclosed subject matter can be formulated as an animal food composition. In particular embodiments, the animal food composition can be fed to dogs according to feeding guidelines.

### 6.2.1. Glycyrrhizin

As used herein, the term “glycyrrhizin” designates a constituent of Glycyrrhiza glabra root, i.e., licorice or liquorice. Glycyrrhizin, the most prominent compound found in licorice, is a triterpene glycoside. Licorice is a perennial plant naturally occurring or cultivated in Europe and Asia. Further, glycyrrhizin has been attributed numerous pharmacological effects like anti-inflammatory, anti-viral, anti-tumor, antioxidant and hepatoprotective activities (Sato et al., 1996; Rahman et al., 2006).

The structure of glycyrrhizin encompasses a triterpene portion (glycyrrhetinic acid) and two iduronic acid residues. Other names for glycyrrhizin include Glycyrrhizinic acid, Glycyrrhizic acid, Glycyrrhetinic acid glycoside, and (3-beta,20-beta)-20-Carboxy-11-oxo-30-norolean-12-en-3-yl-2-O-beta-D-glucopyranuronosyl-alpha-D glucopyranosiduronic acid.

Glycyrrhizin can be extracted from various natural plants by several extraction techniques known by a skilled person in the art, such as, for example, supercritical CO₂ extraction, aqueous two-phase extraction, solvent extraction, third-phase extraction, microwave-assisted extraction, or ultrasound-assisted extraction.

Glycyrrhizin has CAS Registry Number: 1405-86-3 and is characterized by the following structure:

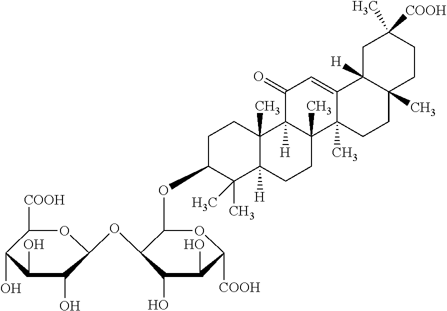

According to one embodiment, glycyrrhizin can be one of its pharmaceutically acceptable salts and/or racemic, enantiomeric, diastereoisomeric or tautomeric forms.

Non-limiting examples of the pharmaceutically acceptable salt are salts with acid or base including ammonium salt such as monoammonium glycyrrhizinate and alkali metal salt such as disodium glycyrrhizinate, trisodium glycyrrhizinate, and dipotassium glycyrrhizinate.

In one embodiment, glycyrrhizin can be found in some naturally-occurring extracts and/or plants, or chemically obtained (Shabkhiz M A, Eikani M H, Bashiri Sadr Z, Golmohammad F. Superheated water extraction of glycyrrhizic acid from licorice root. Food Chem. 2016, 210: 396-401).

According to one embodiment, glycyrrhizin can be extracted from a glycyrrhizin source such as a crude licorice. A glycyrrhizin source can be a glycyrrhizin-containing extract obtained from a plant material. Preferably, a glycyrrhizin-containing extract can be obtained from a plant material containing glycyrrhizin, such as crude licorice, by an extraction method comprising at least the steps of:
(a) Providing a plant material containing glycyrrhizin;
(b) Grinding the plant material of step a), preferably in a solvent, thereby obtained a grinded mixture;
(c) Extracting glycyrrhizin from the grinded mixture, thereby obtaining a glycyrrhizin-containing extract;
(d) Optionally filtering the glycyrrhizin extract, thereby obtained a filtered glycyrrhizin extract;
(e) Optionally concentrating the filtered glycyrrhizin extract;
(f) Optionally spray-drying the filtered glycyrrhizin extract, thereby obtained a powder; and
(g) Optionally sieving the powder.

As used herein a “plant material containing glycyrrhizin” refers to a glycyrrhizin source, preferably to a Glycyrrhiza glabra root, most preferably to a glycyrrhizin-containing licorice extract or a glycyrrhizin-containing crude licorice.

According to some embodiments, the term “plants” or “plant” encompasses glycyrrhiza. In particular embodiments, glycyrrhiza is selected in the group comprising glycyrrhiza acanthocarpa, glycyrrhiza aspera, Glycyrrhiza astragalina, Glycyrrhiza bucharica, Glycyrrhiza echinate, Glycyrrhiza eglandulosa, Glycyrrhiza eurycarpa, glycyrrhiza foetida, Glycyrrhiza foetidissima, Glycyrrhiza frearitis, Glycyrrhiza glabra, glycyrrhiza gontscharovii, Glycyrrhiza iconica, Glycyrrhiza inflata, Glycyrrhiza korshinskyi, glycyrrhiza lepidota, Glycyrrhiza pallidiflora, Glycyrrhiza squamulose, Glycyrrhiza triphylla, glycyrrhiza uralensis and glycyrrhiza yunnanensis.

The term “licorice” or “liquorice” encompasses crude licorice, licorice root and Glycyrrhiza glabra root. In non-limiting embodiments, the glycyrrhizin source consists of a licorice root extract (LRE).

The amount of glycyrrhizin in a given plant source, extract, or food composition, can be expressed in weight per total weight of dry matter of the given plant source, extract or composition.

Licorice can contain from about 1% to about 30%, from about 1% to about 25%, from about 5% to about 20%, from about 6% to about 20%, or from about 6% to about 12% by weight of glycyrrhizin, relative to the total weight of dry matter of the licorice. As used herein, from about 6% to about 12% by weight of glycyrrhizin includes about 6.0%, about 6.5%, about 7.0%, about 7.5%, about 8.0%, about 8.5%, about 9.0%, about 9.5%, about 10.0%, about 10.5%, about 11.0%, about 11.5%, about 12.0% by weight of glycyrrhizin, based on the total weight of dry matter of the licorice root.

Licorice can be provided from a commercially available source such as the Licorice root PE 12% Glycyrrhizin preparation supplied by Naturex (Product ref, ED161596).

In a non-limiting embodiment, the source of glycyrrhizin comprises a licorice, or extract thereof, containing glycyrrhizin. Any other source of glycyrrhizin known by the person skilled in the art, especially any other source of glycyrrhizin apart from licorice, can also be used. According to some embodiments, the glycyrrhizin source can be pure glycyrrhizin. In a non-limiting embodiment, the source of glycyrrhizin comprises a licorice root extract containing 12% glycyrrhizin prepared using a mixture of 80% water and 20% ethanol.

In certain embodiments, the presently disclosed subject matter relates to an animal food composition for use in a method for preventing and/or treating inflammatory diseases or disorders, allergic inflammatory skin diseases wherein the said animal food composition is formulated for providing to an animal a daily amount of glycyrrhizin in an amount of at least about 0.02 mg/kg of bodyweight.

In certain embodiments, the animal food composition is formulated to provide to an animal a daily amount of glycyrrhizin in an amount of from about 0.02 mg/kg to about 2 mg/kg, from about 0.03 mg/kg to about 2 mg/kg, from about 0.05 mg/kg to about 1 mg/kg, from about 0.1 mg/kg to about 0.8 mg/kg, from about 0.2 mg/kg to about 0.6 mg/kg, from about 0.02 mg/kg to about 1 mg/kg, or from about 1 mg/kg to about 2 mg/kg of bodyweight.

As used herein, a daily amount of glycyrrhizin is in an amount of from about 0.02 mg/kg to about 2 mg/kg of bodyweight can include about 0.02 mg/kg, about 0.03 mg/kg, about 0.04 mg/kg, about 0.05 mg/kg, about 0.06 mg/kg, about 0.07 mg/kg, about 0.08 mg/kg, about 0.09 mg/kg, about 0.1 mg/kg, about 0.2 mg/kg, about 0.3 mg/kg, about 0.4 mg/kg, about 0.5 mg/kg, about 0.6 mg/kg, about 0.7 mg/kg, about 0.8 mg/kg, about 0.9 mg/kg, about 1 mg/kg, about 1.1 mg/kg, about 1.2 mg/kg, about 1.3 mg/kg, about 1.4 mg/kg, about 1.5 mg/kg, about 1.6 mg/kg, about 1.7 mg/kg, about 1.8 mg/kg, about 1.9 mg/kg, and about 2 mg/kg of bodyweight.

In another embodiment, the animal food composition is formulated to provide to an animal a daily amount of glycyrrhizin in an amount of from about 0.03 mg/kg to about 2 mg/kg, from about 0.05 mg/kg to about 1 mg/kg, from about 0.1 mg/kg to about 0.8 mg/kg, or about from 0.2 mg/kg to about 0.6 mg/kg of bodyweight. In particular embodiments, the animal food composition is formulated to provide a daily amount of glycyrrhizin of about 0.2 mg/kg of bodyweight, about 0.4 mg/kg of bodyweight or about 0.6 mg/kg of bodyweight.

In certain embodiments, the animal food composition comprising a source of glycyrrhizin can be applied for use as a medicament, wherein the animal food composition is formulated for providing to an animal a daily amount of glycyrrhizin in an amount of at least about 0.02 mg/kg of bodyweight. In certain embodiments, the medicament provides to an animal a daily amount of glycyrrhizin in an amount of from about 0.02 mg/kg to about 2 mg/kg, from about 0.02 mg/kg to about 1 mg/kg, or from about 1 mg/kg to about 2 mg/kg of bodyweight.

In certain embodiments, the animal food composition comprises a source of glycyrrhizin comprising glycyrrhizin in an amount of at least about 5 mg/kg based on the total weight of dry matter of the animal food composition. In certain embodiments, the amount of glycyrrhizin is in an amount of from about 5 mg/kg to about 100 mg/kg based on the total weight of dry matter of the animal food composition. In particular embodiments, the amount of glycyrrhizin is in an amount of from about 5 mg/kg to about 50 mg/kg, from about 5 mg/kg to about 40 mg/kg, from about 10 mg/kg to about 75 mg/kg, from about 15 mg/kg to about 50 mg/kg, from about 20 mg/kg to about 45 mg/kg, or from about 50 mg/kg to about 100 mg/kg based on the total weight of dry matter of the animal food composition.

In certain embodiments, the amount of glycyrrhizin is at least about 5 mg/kg based on the total weight of dry matter of the animal food composition. As used herein, at least about 5 mg/kg based on the total weight of dry matter of the animal food composition can include at least about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, about 20 mg/kg, about 21 mg/kg, about 22 mg/kg, about 23 mg/kg, about 24 mg/kg, about 25 mg/kg, about 26 mg/kg, about 27 mg/kg, about 28 mg/kg, about 29 mg/kg, about 30 mg/kg, about 31 mg/kg, about 32 mg/kg, about 33 mg/kg, about 34 mg/kg, about 35 mg/kg, about 36 mg/kg, about 37 mg/kg, about 38 mg/kg, about 39 mg/kg, or about 40 mg/kg based on the total weight of dry matter of the animal food composition.

In certain embodiments, the amount of glycyrrhizin is less than about 100 mg/kg based on the total weight of dry matter of the animal food composition. As used herein, less than about 100 mg/kg based on the total weight of dry matter of the animal food composition can include less than about 99 mg/kg, about 98 mg/kg, about 97 mg/kg, about 96 mg/kg, about 95 mg/kg, about 94 mg/kg, about 93 mg/kg, about 92 mg/kg, about 91 mg/kg, about 90 mg/kg, about 89 mg/kg, about 88 mg/kg, about 87 mg/kg, about 86 mg/kg, about 85 mg/kg, about 84 mg/kg, about 83 mg/kg, about 82 mg/kg, about 81 mg/kg, about 80 mg/kg, about 79 mg/kg, about 78 mg/kg, about 77 mg/kg, about 76 mg/kg, about 75 mg/kg, about 74 mg/kg, about 73 mg/kg, about 72 mg/kg, about 71 mg/kg, about 70 mg/kg, about 69 mg/kg, about 68 mg/kg, about 67 mg/kg, about 66 mg/kg, about 65 mg/kg, about 64 mg/kg, about 63 mg/kg, about 62 mg/kg, about 61 mg/kg, about 60 mg/kg, about 59 mg/kg, about 58 mg/kg, about 57 mg/kg, about 56 mg/kg, about 55 mg/kg, about 54 mg/kg, about 53 mg/kg, about 52 mg/kg, about 51 mg/kg, or about 50 mg/kg based on the total weight of dry matter of the animal food composition.

### 6.2.2. Additional Ingredients

In certain embodiments, the animal food composition for preventing and/or treating inflammatory diseases or disorders, allergic inflammatory skin diseases further comprises linoleic acid.

In particular embodiments, the animal food composition for preventing and/or treating inflammatory diseases or disorders, allergic inflammatory skin diseases disclosed herein further comprises one or more curcuminoids.

In certain embodiments, the animal food composition further comprises EPA/DHA, Taurine, Lutein, Vitamin E and/or a combination thereof. The animal food composition can, in some embodiments, additionally further comprise one or more vitamin A, vitamin B₃, vitamin C, zinc, vitamin D, one or more fatty acids, and/or a combination thereof.

### 6.2.2.1. Curcuminoids

As used herein, the term “curcuminoid” or “curcuminoids” refers to phenols that are present in the Indian spice turmeric. Turmeric is generally derived from the roots of the plant Curcuma longa. Curcuminoids have also been found in roots of other species in the plant family Zingiberaceae of the Curcuma genus. In particular, turmeric can contain from about 60% to about 80% in weight of curcumin, relative to the total weight of dry matter of turmeric, from about 15% to about 30% in weight of demethoxycurcumin, relative to the total weight of dry matter of turmeric, and from about 2% to about 6% in weight of bis-demethoxycurcumin, relative to the total weight of dry matter of turmeric. The curcuminoid in the food composition of the presently disclosed subject matter can be of any format, including a powder or lipid extract. In a non-limiting embodiment, curcuminoids can be found in some naturally-occurring extracts and/or plants, or can be chemically obtained or synthesized.

In non-limiting embodiments, the source of curcuminoids comprises a turmeric extract, i.e., Curcuma Longa. In certain embodiments, the turmeric extract can be BCM-95® from Arjuna or turmeric extract from Naturex. Any other source of curcuminoids known by the person skilled in the art can also be used.

Other available sources for curcuminoids can also be selected from liposomal curcumin, curcumin nanoparticles, curcumin phospholipid complex (e.g., MERIVA sourcing, about 20% total curcuminoids to BCM-95® Arjuna with about 90% in weight of total curcuminoids), structural analogues of curcumin (e.g., EF-24) demethoxycurcumin, bisdemethoxycurcumin, tetrahydrocurcumin, and commercial/DM, any formulation designed to enhance curcumin bioavailability.

Typically, curcuminoids can be found in other botanicals in addition to Curcuma longa, such as Curcuma xanthorrhiza and Curcuma zedoania. Curcuminoid in its pure form has poor solubility in water. Curcuminoids can be extracted from curcuminoids-containing plants, i.e., turmeric root, with organic solvent such as ethanol or acetone.

In particular embodiments, curcuminoids according to the presently disclosed subject matter can comprise compounds of the general formula (I):

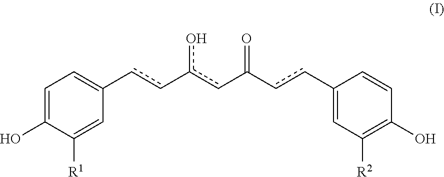

wherein R¹ and R² are independently chosen among hydrogen atom, methoxy, methyl, hydroxyl and ethoxy group, or one of its pharmaceutically acceptable salts and/or racemic, enantiomeric, diastereoisomeric or tautomeric forms.

In non-limiting embodiments, R¹ and R² can be the same or different.

In non-limiting embodiments, the curcuminoids are selected in the group consisting of curcumin, demethoxycurcumin, bis-methoxycucurmin, tetrahydrocurcumin, or a combination thereof.

For reference, curcumin (I), demethoxycurcumin (II), bis-methoxycurcumin (III) and tetrahydrocurcumin (IV) consist of the chemical structures:

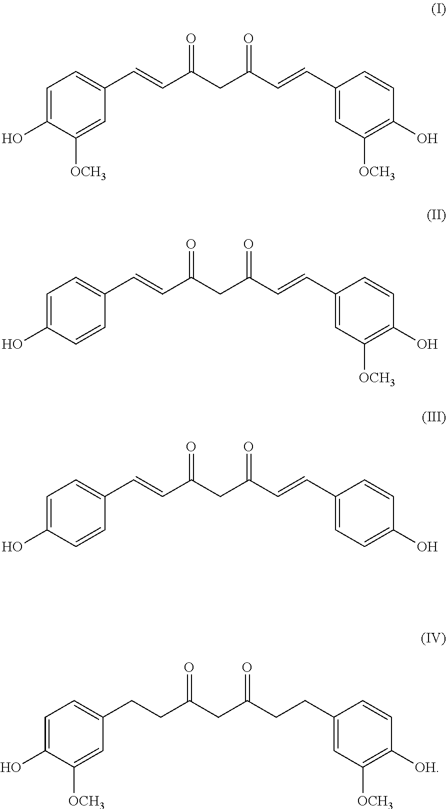 [SMILES: COc1cc(/C=C/C(=O)CC(=O)/C=C/c2ccc(O)c(OC)c2)ccc1O ; COc1cc(/C=C/C(=O)CC(=O)/C=C/c2ccc(O)cc2)ccc1O ; O=C(/C=C/c1ccc(O)cc1)CC(=O)/C=C/c1ccc(O)cc1 ; COc1cc(CCC(=O)CC(=O)CCc2ccc(O)c(OC)c2)ccc1O]

In certain embodiments, the curcuminoids is curcumin. In particular embodiments, the source of curcuminoids (i.e., curcumin) can be turmeric.

In certain embodiments, the animal food composition comprises a source of curcuminoids in an amount ranging from about 0.01 g/Mcal to about 0.1 g/Mcal, from about 0.01 g/Mcal to about 2 g/Mcal, from about 0.01 g/Mcal to about 5 g/Mcal, from about 5 g/Mcal to about 10 g/Mcal, or from about 0.01 g/Mcal to about 10 g/Mcal. In certain embodiments, the animal food composition comprises a source of curcuminoids in an amount of less than about 5 g/Mcal, less than about 2 g/Mcal, or less than about 0.5 g/Mcal.

In particular embodiments, the animal food composition comprises a source of curcuminoids in an amount ranging from about 0.01% to about 0.1%, from about 0.01% to about 5%, from about 5% to about 10%, from about 0.1% to about 10%, or from about 0.01% to about 10% by weight, based on the total weight of dry matter of the food composition.

As used herein, from about 0.01% to about 10% by weight of a source of curcuminoids can include about 0.01%, about 0.1%, about 1%, about 2%, about 3%, about 4%, about 5%, about 6%, about 7%, about 8%, about 9% or about 10% by weight of a source of curcuminoids, based on the total weight of dry matter of the food composition.

As used herein, from about 0.01% to about 0.1% of a source of curcuminoids can include about 0.01%, about 0.011%, about 0.012%, about 0.013%, about 0.014%, about 0.015%, about 0.016%, about 0.017%, about 0.018%, about 0.019%, about 0.02%, about 0.021%, about 0.022%, about 0.023%, about 0.024%, about 0.025%, about 0.026%, about 0.027%, about 0.028%, about 0.029%, about 0.03%, about 0.031%, about 0.032%, about 0.033%, about 0.034%, about 0.035%, about 0.036%, about 0.037%, about 0.038%, about 0.039%, about 0.04%, about 0.041%, about 0.042%, about 0.043%, about 0.044%, about 0.045%, about 0.046%, about 0.047%, about 0.048%, about 0.049%, about 0.05%, about 0.051%, about 0.052%, about 0.053%, about 0.054%, about 0.055%, about 0.056%, about 0.057%, about 0.058%, about 0.059%, about 0.06%, about 0.061%, about 0.062%, about 0.063%, about 0.064%, about 0.065%, about 0.066%, about 0.067%, about 0.068%, about 0.069%, about 0.07%, about 0.071%, about 0.072%, about 0.073%, about 0.074%, about 0.075%, about 0.076%, about 0.077%, about 0.078%, about 0.079%, about 0.08%, about 0.081%, about 0.082%, about 0.083%, about 0.084%, about 0.085%, about 0.086%, about 0.087%, about 0.088%, about 0.089%, about 0.09%, about 0.091%, about 0.092%, about 0.093%, about 0.094%, about 0.095%, about 0.096%, about 0.097%, about 0.098%, about 0.099%, about 0.1% of a source of curcuminoids by weight, based on the total weight of dry matter of the animal food composition.

In certain embodiments, the curcuminoid source can consist of pure curcuminoids.

In non-limiting examples, an animal food composition, in particular in the form of dry food, suitable for a small dog (see the classification provided in the specification) can comprise about 0.002 wt % of glycyrrhizin and about 0.0275 wt % of curcuminoids relative to the total weight of the composition. According to another example, an animal food composition, in particular in the form of dry food, suitable for a medium size or large dog (see the classification provided in the specification) can comprise about 0.003 wt % of glycyrrhizin and about 0.0275 wt % of curcuminoids relative to the total weight of the composition. An animal food composition as described herein can be formulated for providing to an animal a daily amount of curcuminoids of about 77 mg/kg, in particular in the form of wet food.

### 6.2.2.2. Linoleic Acid

As used herein, the term “linoleic acid” means a polyunsaturated omega-6 fatty acid, which is one of two essential fatty acids for animals.

For measuring the content of a food composition in linoleic acid, the one skilled in the art can refer to any of well-known techniques. As an example, a method based upon the norm NF EN ISO 5508/5509 via chromatography in gaseous phase can be used. Thus, in some embodiments, the animal food composition described herein comprises one or more substances which are sources of linoleic acid.

Linoleic acid sources, i.e., linoleic acid-containing substances, can include vegetable oils, although animal oils or crude fats can also be used. Linoleic sources can include safflower oil, sunflower oil, soybean oil, sesame oil, canola oil, other plant or animal oils/crude fats, meats or a combination of two or more thereof. In certain embodiments, linoleic acid sources are selected from the group consisting of safflower oil, sunflower oil, soybean oil, sesame oil, canola oil, meats, and combinations thereof.

In certain embodiments, the animal food composition comprises linoleic acid in an amount ranging from about 1 g/Mcal to about 20 g/Mcal, from about 1 g/Mcal to about 10 g/Mcal, from about 8 g/Mcal to about 12 g/Mcal, from about 10 g/Mcal to about 12 g/Mcal, or from about 10 g/Mcal to about 20 g/Mcal.

In certain embodiments, the animal food composition comprises linoleic acid in an amount ranging from about 2% to about 10%, from about 2% to about 5.5%, or from about 5.5% to about 10% by weight relative to the total weight of dry matter of the animal food composition.

### 6.2.2.3. EPA/DHA

As used herein, the term “EPA/DHA” designates a fatty acid or a mixture of fatty acids consisting of (i) only eicosapentaenoic acid ethyl ester (EPA), (ii) only docosahexaenoic acid ethyl ester (DHA) or (iii) a combination of eicosapentaenoic acid ethyl ester and docosahexaenoic acid ethyl ester (EPA+DHA). Thus, as used herein, an amount of “EPA/DHA” means (i) an amount of EPA, in the absence of DHA, (ii) an amount of DHA, in the absence of EPA, or (iii) an amount of a combination of EPA and DHA.

In certain embodiments, the animal food composition comprises a combination of Eicosapentaenoic acid (EPA) and Docosahexaenoic acid (DHA) in a weight ratio of EPA:DHA from about 0.0001:1000 to about 1000:0.0001. In certain embodiments, the weight ratio of EPA:DHA is about 0.0001:100, about 1:1, or about 0.1:0.0001, relative to the total weight of dry matter of the animal food composition. In particular embodiments, the weight ratio of EPA:DHA can be about 0.1:100, about 100:0.1, or from 0.1 to 100. In 5 particular embodiments, the weight or energy ratio of EPA to DHA may range from 0.0001 to 1000 relative to the total weight of the dry matter of the animal food composition.

According to some embodiments, the animal food composition comprises EPA/DHA in an amount of from about 0.1 g/Mcal to about 7 g/Mcal, from about 0.1 g/Mcal to about 3 g/Mcal, from about 1 g/Mcal to about 2 g/Mcal, from about 1.25 g/Mcal to about 1.75 g/Mcal, or from about 3 g/Mcal to about 7 g/Mcal. In certain embodiments, the animal food composition comprises EPA/DHA in an amount of from about 0.1% to about 5%, from about 0.1% to about 2.5%, or about 2.5% to about 5% by weight, relative to the total weight of dry matter of the animal food composition.

### 6.2.2.4. Taurine

Taurine is a non-essential amino acid which is obtained from meat and fish. It stimulates the production of glycosphingolipids in the skin by acting as a precursor molecule. Glycosphingolipids exhibit anti-microbial properties.

Taurine, according to the presently disclosed subject matter can have the following structure:

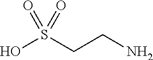

or one of its pharmaceutically acceptable salts and/or racemic, enantiomeric, diastereoisomeric or tautomeric forms.

In certain embodiments, the animal food composition comprises taurine in an amount of from about 1 g/Mcal to about 2 g/Mcal, from about 1 g/Mcal to about 1.5 g/Mcal, or from about 1.5 g/Mcal to about 2 g/Mcal.

### 6.2.2.5. Lutein

As used herein, “lutein” refers to a xanthophyll and is one of the known naturally occurring carotenoids. Lutein is a lipophilic molecule and is generally insoluble in water. Lutein is synthesized only by plants and like other xanthophylls is found in high quantities in green leafy vegetables such as spinach, kale and yellow carrots. Lutein is isomeric with zeaxanthin, differing only in the placement of one double bond. The principal natural stereoisomer of lutein is (3R,3′R,6′R)-beta,epsilon-carotene-3,3′-diol. Lutein is present in plants as fatty-acid esters, with one or two fatty acids bound to the two hydroxyl-groups.

Lutein according to the presently disclosed subject matter can have the following structure:

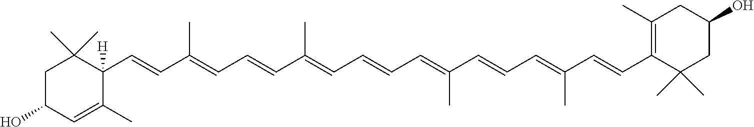 [SMILES: CC1=C[C@H](O)CC(C)(C)[C@H]1/C=C/C(C)=C/C=C/C(C)=C/C=C/C=C(C)/C=C/C=C(C)/C=C/C1=C(C)C[C@@H](O)CC1(C)C]

or one of its pharmaceutically acceptable salts and/or racemic, enantiomeric, diastereoisomeric or tautomeric forms.

In certain embodiments, the animal food composition comprises lutein in an amount of from about 0.001 g/Mcal to about 0.002 g/Mcal, from about 0.001 g/Mcal to about 0.0015 g/Mcal, or from about 0.0015 g/Mcal to about 0.002 g/Mcal.

### 6.2.2.6. Vitamin E

Vitamin E is a collective term for several biologically similar compounds, including those called tocopherols and tocotrienols, which share the same biological activity. The most biologically active biological form of vitamin E (also the most active antioxidant) in animal tissue is alpha-tocopherol. Vitamin E cannot be synthesized in vivo. Vitamin E protects against the loss of cell membrane integrity, which adversely alters cellular and organelle function. Vitamin E according to an aspect of the presently disclosed subject matter can be in any form. It can be liquid, semi-solid or solid. It can be a tocopherol or a tocotrienol. It can be alphatocopherol, (d-x or dl-oc) beta-tocopherol (d-,B or dl-), gamma-tocopherol (dot or dim), delta-tocopherol, alpha-tocotrienol, beta-tocotrienol, gamma-tocotrienol or delta-tocotrienol. In particular embodiments, it is alpha-tocopherol.

In certain embodiments, the source of the vitamin E is not limiting. In non-limiting embodiments, vitamin E sources include vitamin E acetate, (e.g., tocopherol acetate), vitamin E acetate adsorbate, or vitamin E acetate spray dried. In certain embodiments, sources are synthetic although natural sources can be used.

For reference, vitamin E according to the presently disclosed subject matter can have the following structure:

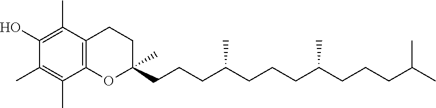 [SMILES: Cc1c(C)c2c(c(C)c1O)CC[C@@](C)(CCC[C@H](C)CCC[C@H](C)CCCC(C)C)O2]

or one of its pharmaceutically acceptable salts and/or racemic, enantiomeric, diastereoisomeric or tautomeric forms.

In certain embodiments, the animal food composition comprises vitamin E in an amount of from about 0.2 g/Mcal to about 0.3 g/Mcal, from about 0.2 g/Mcal to about 0.25 g/Mcal, or from about 0.25 g/Mcal to about 0.3 g/Mcal.

### 6.2.3. Additives

In some embodiments, the animal food composition as described herein is nutritionally complete and can also contain additives, for example, protein, crude fat, crude fibers, nitrogen free extract, ash, minerals, vitamins or condiments.

As it shall be readily understood, every embodiment of an animal food composition encompassed by the presently disclosed subject matter comprises a variety of ingredients, each comprised in the said composition at a given weight percentage, as compared to the total weight of dry matter of the animal food composition.

An animal food composition as disclosed herein comprises, protein, crude fat, ash, fiber, NFE and optionally one or more further additives such as vitamins, minerals, etc., the sum of the weight of each of the ingredients comprised therein amounting to 100% by weight, based on the total weight of dry matter of the said animal food composition.

### 6.2.3.1. Protein

In certain embodiments, an animal food composition according to the presently disclosed subject matter can further contain a protein source. Accordingly, the protein level shall be high enough so as to ensure maintenance of lean body mass. An animal food composition can contain one or more distinct protein sources.

In particular embodiments, an animal food composition as described herein can comprise a plurality of proteins that are contained in a protein source which is used in the manufacture process. In certain embodiments, the animal food composition as described herein can comprise a plurality of proteins from a plurality of protein sources.

In certain embodiments, the protein is not hydrolyzed. In some other embodiments a protein can be present in an at least partially hydrolysed form, or even completely hydrolyzed. An animal food composition according to the presently disclosed subject matter can incorporate proteins in the form of meat or animal derived material such as beef, chicken, turkey, lamb, fish, blood plasma, marrow bone, or combinations thereof. In certain embodiments, an animal food composition as described herein can be meat-free. In non-limiting embodiments, the meat-free animal food composition comprises a meat substitute protein source such as soya, maize gluten or any other protein-containing soya product in order to provide a protein source. An animal food composition as disclosed herein can comprise additional protein sources such as soya protein concentrate, milk proteins, gluten, etc.

### 6.2.3.2. Crude Fat

An animal food composition according to the present disclosure can further contain a nutritionally appropriate amount of crude fat. The expression “crude fat” as used in the present specification comprises any food-acceptable crude fat(s) and/or oil(s) irrespective of their consistency at room temperature, i.e., irrespective whether said “crude fat” is present in essentially fluid form or in essentially solid form. The animal food composition according to the presently disclosed subject matter can comprise crude fat of animal and/or vegetable origin. Crude fat can be supplied by any of a variety of sources known by those skilled in the art. Plant crude fat sources include, without limitation, wheat, sunflower, safflower, rapeseed, olive, borage, flaxseed, peanuts, blackcurrant seed, cottonseed, wheat, germ, corn germ as well as oils derived from these and other plant crude fat sources. Animal sources can include, for example and without limitation, chicken crude fat, turkey crude fat, beef crude fat, duck crude fat, pork crude fat, lamb crude fat, fish oil or any meat, meat by-products, seafood, dairy, eggs, etc. Crude fat content of foods can be determined by any number of methods known by those of skill in the art.

### 6.2.3.3. Fibers

Fibers are optionally included in an animal food composition disclosed herein. The expression “fibers” is similar to “dietary fibers” and shall be interpreted for the purpose of the presently disclosed subject matter as Total Fibers, meaning that it includes soluble fibers and insoluble fibers. Soluble fiber can be defined as being resistant to digestion and absorption in the small intestine and undergo complete or partial fermentation in the large intestine by opposition to insoluble fiber that can be defined as non-starch polysaccharides that are resistant to digestion and absorption in the small intestine, and resistant to fermentation in the large intestine. Soluble fibers are considered as having a prebiotic effect by providing short chain fatty acids as a source of energy to colonocytes. Insoluble fibers are considered useful for transit and ballast effect. Non-limiting examples of fibers include beet pulp, guar gum, chicory root, psyllium, pectin, blueberry, cranberry, squash, apples, oats, beans, citrus, barley, or peas, and a second group comprising cellulose, whole wheat products, wheat oat, corn bran, flax seed, grapes, celery, green beans, cauliflower, potato skins, fruit skins, vegetable skins, peanut hulls, and soy fiber.

### 6.2.3.4. Nitrogen Free Extract (NFE)

As used herein, and as conventionally understood in the art, the Nitrogen Free Extract (NFE) consists of the soluble carbohydrate fraction that can be optionally included in an animal food composition disclosed herein. NFE can encompass soluble polysaccharides, starch, gums, mucilages, and pectin, if present in the said animal food composition. Thus, as it is conventionally known in the art, NFE does not comprise the insoluble carbohydrate fraction in the crude fiber material that can, in certain embodiments, be present in the said food composition. Typically, the content of Nitrogen Free Extract in the food composition is determined by subtracting the content of each of the other components (protein, crude fat, crude fiber, ash) from the whole dry matter of the said food composition. In embodiments wherein the qualitative and quantitative features of a food composition is expressed as the energy density (e.g., the metabolizable energy density in g/Mcal), the NFE content is determined by subtracting the energy value of each of the other components (protein, crude fat, crude fiber, ash) from the energy value of the whole food composition. In embodiments wherein the qualitative and quantitative features of a food composition is expressed as a weight percentage (e.g., a weight percentage based on the total weight of dry matter of the said composition), the NFE content is determined by subtracting the weight percentage of the other components (protein, crude fat, crude fiber, ash) from the total weight of the said food composition.

### 6.2.3.5. Carbohydrates

As used herein, the term “carbohydrates” designates a mixture of polysaccharides and sugars that are metabolized for energy when hydrolyzed in the body. The carbohydrate content of foods can be determined by any number of methods known by those of skilled in the art. Carbohydrates can be supplied as a variety of carbohydrate sources known by those skilled in the art, including starch (e.g., any kinds, corn, wheat, barley), beet pulp (which contain a bit of sugars) and psyllium.

### 6.2.3.6. Starch

An important source of Nitrogen Free Extract that is optionally included in an animal food composition disclosed herein contains starch. The term “starch” as used herein refers to a polysaccharide that is composed of amylose and amylopectin. Starch occurs in many plant tissues as granules, usually between 1 and 100 μm in diameter, depending upon the plant source. Chemically, starches are polysaccharides composed of α-D-glucopyranosyl units linked together with α-D(1-4) and/or α-D(1-6) linkages and are comprised of two molecular types: amylose, the straight chain polyglucan comprised of approximately 1000, α-D(1-4) linked glucoses; and amylopectin, the branched glucan, comprised of approximately 4000 glucose unite with branches occurring as α-D(1-6) linkages. As used herein, starch encompasses the various crystalline structures of A-type, B-type and C-type starches, which contain different proportions of amylopectin. A-type starches are found mainly in cereals, while B-type starches are found mainly in tubers and amylose-rich starches. C-type starch consists of a mixture of both A and B forms and is found mainly in legumes. In general, digestible starches are broken down (hydrolyzed) by the enzymes a-amylases, glucoamylase and sucrose-iso-maltase in the small intestine to yield free glucose that is then absorbed. Starch comprised in an animal food disclosed herein can consist in any starch suitable for dietary purpose. Indeed, the native starch comprised in the starting materials used for preparing an animal food composition as described herein is susceptible to undergo changes during the manufacture process. The amount of starch in the animal food composition comprises the amount of starch that is contained in the total amount of the raw materials of the animal food composition. However, the amount of starch comprised in an animal food composition disclosed herein equates the total amount of starch comprised in the starting materials used for preparing the said animal food composition. If not already known, the starch content of the starting materials used for preparing an animal food composition described herein can be determined according to conventional techniques known in the art, and especially according to the known polarimetric method, such as according to NF EN ISO 10520. In some embodiments where the starting materials can contain modified starch or pre-gelatinized starch. The starch content can be determined according to NF EN ISO 15914.

### 6.2.3.7. Ash

The ash content, if specified as a result of an analytical measure of an animal food composition described herein, is a measure of the total amount of minerals comprised herein. The mineral content is a measure of the amount of specific inorganic components comprised herein, which includes calcium (Ca), sodium (Na), potassium (K) and chlorine (Cl).

### 6.2.4. Animal Food Composition Formulations

The presently disclosed subject matter further relates to an animal food composition comprising a source of glycyrrhizin, wherein the amount of glycyrrhizin is higher than 5 mg/kg based on the total weight of dry matter of the animal food composition. The animal food compositions described herein can be as described above.

According to one embodiment, the amount of glycyrrhizin is from about 5 mg/kg to about 100 mg/kg based on the total weight of dry matter of the animal food composition.

In certain embodiments, the amount of glycyrrhizin can be from about 5 mg/kg to about 100 mg/kg, from about 10 mg/kg to about 75 mg/kg, from about 15 mg/kg to about 50 mg/kg, or from about 20 mg/kg to about 45 mg/kg based on the total weight of dry matter of the animal food composition.

In certain embodiments, a source of glycyrrhizin can contain at least one of the plants of genus Glycyrrhiza, preferably of the Glycyrrhiza glabra root, i.e., licorice. In certain embodiments, a source of glycyrrhizin is licorice. In particular embodiments, the source of glycyrrhizin in the animal food composition comprises a licorice root extract.

In particular embodiments, a source of glycyrrhizin is present in an amount ranging from about 0.01 g/Mcal to about 0.1 g/Mcal, based on the total weight of dry matter of the animal food composition. In particular embodiments, a source of glycyrrhizin is present in an amount ranging from about 0.01% to about 10% by weight, based on the total weight of dry matter of the animal food composition.

In certain embodiments, the animal food composition is a nutritionally complete animal food composition, a food supplement, or a topper composition. In some embodiments, an animal food composition consists of a kibble. In particular embodiments, the animal food composition comprises a nutritionally complete animal food composition. In particular embodiments, the animal food composition is formulated for oral administration. In certain embodiments, the animal food composition comprises a food supplement and/or a drug.

In particular embodiments, the animal food composition as described herein further comprises linoleic acid. In certain embodiments, the source of linoleic acid is selected from the group consisting of safflower oil, sunflower oil, soybean oil, sesame oil, canola oil, meats, and combinations thereof.

In certain embodiments, the animal food composition further comprises curcuminoids, linoleic acid, EPA/DHA, Taurine, Lutein, Vitamin E and/or a combination thereof. In certain embodiments, the animal food composition further comprises EPA/DHA, Taurine, Lutein, vitamin E and/or a combination thereof. In some embodiments, an animal food composition further comprising a source of proteins.

In certain embodiments, the animal food composition comprises a source of glycyrrhizin. In certain embodiments, the animal food composition comprises a source of glycyrrhizin and curcuminoids. In certain embodiments, the animal food composition comprises a source of glycyrrhizin and linoleic acid. In certain embodiments, the animal food composition comprises a source of glycyrrhizin and EPA/DHA. In certain embodiments, the animal food composition comprises a source of glycyrrhizin and taurine. In certain embodiments, the animal food composition comprises a source of glycyrrhizin and lutein. In certain embodiments, the animal food composition comprises a source of glycyrrhizin and vitamin E. In certain embodiments, the animal food composition comprises a source of glycyrrhizin, curcuminoids, and linoleic acid. In certain embodiments, the animal food composition comprises a source of glycyrrhizin, curcuminoids, and EPA/DHA. In certain embodiments, the animal food composition comprises a source of glycyrrhizin, curcuminoids, and taurine. In certain embodiments, the animal food composition comprises a source of glycyrrhizin, curcuminoids, and lutein. In certain embodiments, the animal food composition comprises a source of glycyrrhizin, curcuminoids, and vitamin E. In certain embodiments, the animal food composition comprises a source of glycyrrhizin, linoleic acid, and EPA/DHA. In certain embodiments, the animal food composition comprises a source of glycyrrhizin, linoleic acid, and taurine. In certain embodiments, the animal food composition comprises a source of glycyrrhizin, linoleic acid, and lutein. In certain embodiments, the animal food composition comprises a source of glycyrrhizin, linoleic acid, and vitamin E. In certain embodiments, the animal food composition comprises a source of glycyrrhizin, EPA/DHA, and taurine. In certain embodiments, the animal food composition comprises a source of glycyrrhizin, EPA/DHA, and lutein. In certain embodiments, the animal food composition comprises a source of glycyrrhizin, EPA/DHA, and vitamin E. In certain embodiments, the animal food composition comprises a source of glycyrrhizin, taurine, and lutein. In certain embodiments, the animal food composition comprises a source of glycyrrhizin, taurine, and vitamin E. In certain embodiments, the animal food composition comprises a source of glycyrrhizin, lutein and vitamin E. In certain embodiments, the animal food composition comprises a source of glycyrrhizin, curcuminoids, linoleic acid, and EPA/DHA. In certain embodiments, the animal food composition comprises a source of glycyrrhizin, curcuminoids, linoleic acid, and taurine. In certain embodiments, the animal food composition comprises a source of glycyrrhizin, curcuminoids, linoleic acid, and lutein. In certain embodiments, the animal food composition comprises a source of glycyrrhizin, curcuminoids, linoleic acid, and vitamin E. In certain embodiments, the animal food composition comprises a source of glycyrrhizin, curcuminoids, EPA/DHA, taurine, and lutein. In certain embodiments, the animal food composition comprises a source of glycyrrhizin, curcuminoids, EPA/DHA, taurine, and vitamin E. In certain embodiments, the animal food composition comprises a source of glycyrrhizin, EPA/DHA, taurine, lutein, and vitamin E. In certain embodiments, the animal food composition comprises a source of glycyrrhizin, curcuminoids, linoleic acid, EPA/DHA, taurine, and lutein. In certain embodiments, the animal food composition comprises a source of glycyrrhizin, curcuminoids, linoleic acid, EPA/DHA, taurine, and vitamin E. In certain embodiments, the animal food composition comprises a source of glycyrrhizin, linoleic acid, EPA/DHA, taurine, lutein, and vitamin E. In certain embodiments, the animal food composition comprises a source of glycyrrhizin, curcuminoids, linoleic acid, EPA/DHA, taurine, lutein, and vitamin E.

In particular embodiments, the animal food composition of the presently disclosed subject matter comprises licorice in an amount to provide a daily amount of glycyrrhizin in an amount of from about 0.02 mg/kg to about 2 mg/kg of bodyweight. In certain embodiments, the animal food composition further comprises, curcuminoids in an amount of from about 0.01 g/Mcal to about 10 g/Mcal, linoleic acid in an amount of from about 1 g/Mcal to about 20 g/Mcal, EPA/DHA in an amount of from about 0.1 g/Mcal to about 7 g/Mcal, taurine in an amount of from about 1 g/Mcal to about 2 g/Mcal, lutein in an amount of from about 0.001 g/Mcal to about 0.002 g/Mcal, vitamin E in an amount of from about 0.2 g/Mcal to about 0.3 g/Mcal, or combinations thereof.

In particular embodiments, the animal food composition of the presently disclosed subject matter comprises licorice in an amount to provide a daily amount of glycyrrhizin in an amount of from about 0.02 mg/kg to about 1 mg/kg of bodyweight. In certain embodiments, the animal food composition further comprises, curcuminoids in an amount of from about 0.01 g/Mcal to about 5 g/Mcal, linoleic acid in an amount of from about 10 g/Mcal to about 20 g/Mcal, EPA/DHA in an amount of from about 0.1 g/Mcal to about 3 g/Mcal, taurine in an amount of from about 1 g/Mcal to about 1.5 g/Mcal, lutein in an amount of from about 0.001 g/Mcal to about 0.0015 g/Mcal, vitamin E in an amount of from about 0.2 g/Mcal to about 0.25 g/Mcal, or combinations thereof.

In particular embodiments, the animal food composition of the presently disclosed subject matter comprises licorice in an amount to provide a daily amount of glycyrrhizin in an amount of at least about 0.02 mg/kg of bodyweight. In certain embodiments, the animal food composition further comprises linoleic acid in an amount of from about 8 g/Mcal to about 12 g/Mcal, EPA/DHA in an amount of from about 1 g/Mcal to about 2 g/Mcal, taurine in an amount of from about 1 g/Mcal to about 2 g/Mcal, lutein in an amount of from about 0.001 g/Mcal to about 0.002 g/Mcal, vitamin E in an amount of from about 0.2 g/Mcal to about 0.3 g/Mcal, or combinations thereof.

In particular embodiments, the animal food composition of the presently disclosed subject matter comprises licorice in an amount to provide a daily amount of glycyrrhizin in an amount of at least about 0.02 mg/kg of bodyweight. In certain embodiments, the animal food composition further comprises linoleic acid in an amount of from about 10 g/Mcal to about 12 g/Mcal, EPA/DHA in an amount of from about 1.25 g/Mcal to about 1.75 g/Mcal, taurine in an amount of from about 1 g/Mcal to about 1.5 g/Mcal, lutein in an amount of from about 0.001 g/Mcal to about 0.0015 g/Mcal, vitamin E in an amount of from about 0.2 g/Mcal to about 0.25 g/Mcal, or combinations thereof.

In particular embodiments, the animal food composition of the presently disclosed subject matter comprises licorice in an amount to provide a daily amount of glycyrrhizin in amount of at least about 0.02 mg/kg of bodyweight, linoleic acid in an amount of about 10.5 g/Mcal, EPA/DHA in an amount of about 1.5 g/Mcal, taurine in an amount of about 1.2 g/Mcal, lutein in an amount of about 0.0013 g/Mcal, vitamin E in an amount of about 0.244 g/Mcal.

In certain embodiments, the animal food composition further comprises sources of protein, crude fat, fibers, NFE, carbohydrates, starch, or combinations thereof.

In certain embodiments, the animal is a canine. In particular embodiments, the canine is a dog. In particular embodiments, the animal food composition comprises or consists of dry, wet and/or semi-moist food compositions.

The presently disclosed subject matter further provides a kit as described herein. The kit comprises:
(i) a first part comprising a source of glycyrrhizin; and
(ii) a second part comprising one or more additional ingredients;
   for use in a method for preventing and/or treating inflammatory diseases or disorders, allergic inflammatory skin diseases, wherein the kit is formulated for providing to an animal a daily amount of glycyrrhizin in an amount of at least about 0.02 mg/kg of bodyweight. In certain embodiments, the kit comprises glycyrrhizin and one or more additional ingredients as disclosed herein.

In certain embodiments, a source of glycyrrhizin can contain of at least one of the plants of genus Glycyrrhiza, preferably of the Glycyrrhiza glabra root, i.e., licorice. In certain embodiments, a source of glycyrrhizin is licorice. In particular embodiments, the source of glycyrrhizin in the kit comprises a licorice root extract.

In particular embodiments, the kit contains dry, wet and/or semi-moist food compositions. In certain embodiments, the kit contains a nutritionally complete animal food composition, a food supplement, or a topper composition. In some embodiments, the kit contains a kibble. In one embodiment, the kit is formulated for oral administration. In one embodiment the kit contains a food supplement and/or a drug.

### 6.2.5. Dog Breeds and Feeding Guidelines

The present disclosure relates to, inter alia, methods for assessing health and well-being of animals. Characteristics of companion animals can vary, including by size, sex, breed, and species. Given that domesticated animals receive the majority of veterinary care, examined trends and individual animal characteristics for the most common domesticated animal (e.g., dogs) can generally provide an indication of the efficacy of the examined method when applied to other animals.

The present disclosure relates to, inter alia, methods to determine oral biometrics of an animal of the genus Canis. The Canis genus comprises domestic dogs (Canis lupus familiaris), wolves, coyotes, foxes, jackals, dingoes and the present disclosure can be used for all these animals. In some embodiments, the subject is a domestic dog, herein referred to simply as a dog. This Canis genus is part of the Canidae family, which includes numerous extant species.

As used herein, the expression “size category” refers to the definition of the animal (e.g., dogs, cats, etc.) in terms of the average weight of the particular animal breed. Animals (e.g., dogs, cats, etc.) of the same breed can have relatively uniform physical characteristics, such as size, coat color, physiology, and behavior, as compared to animals of a different breed. It is noted that the discussion below is focused on dogs, however, other companion animals and wild animals are intended to be covered by the scope of this disclosure and the present disclosure is not intended to be limited to dogs.

The dog can be any breed of dog, including toy, small, medium, large or giant breeds. Non-limiting examples of toy breeds include Affenpinscher, Australian Silky Terrier, Bichon Frise, Bolognese, Cavalier King Charles Spaniel, Chihuahua, Chinese Crested, Coton De Tulear, English Toy Terrier, Griffon Bruxellois, Havanese, Italian Greyhound, Japanese Chin, King Charles Spaniel, Lowchen (Little Lion Dog), Maltese, Miniature Pinscher, Papillon, Pekingese, Pomeranian, Pug, Russian Toy, and Yorkshire Terrier. Examples of small breeds include, but are not limited to, French Bulldog, Beagle, Dachshund, Pembroke Welsh Corgi, Miniature Schnauzer, Cavalier King Charles Spaniel, Shih Tzu, and Boston Terrier. Examples of medium dog breeds include, but are not limited to, Bulldog, Cocker Spaniel, Shetland Sheepdog, Border Collie, Basset Hound, Siberian Husky, Dalmatian, Doberman, Staffordshire Bull Terrier. Examples of large breed dogs include, but are not limited to, Bearded Collie, Great Dane, Neapolitan mastiff, Scottish Deerhound, Dogue de Bordeaux, Newfoundland, English mastiff, Saint Bernard, Leonberger, and Irish Wolfhound. Cross-breeds can generally be categorized as toy, small, medium, and large dogs depending on their body weight. In certain embodiments, the dog is a toy breed. In certain embodiments, the dog is a medium, large or giant breed. In some embodiments, the dog is a mix of two or more breeds. In such instances, the mixed-breed dog can still be categorized by size depending on their body weight and may exhibit traits (e.g., behavioral traits, genetic traits, etc.) associated with each of the two or more breeds found in the dog.

The Fédération Cynologique Internationale currently recognizes 346 pure dog breeds. The breed of a dog can be identified, for example, either by observing its physical traits or by genetic analysis. A pedigree dog is the offspring of two dogs of the same breed, which is eligible for registration with a recognized club or society that maintain a register for dogs of that description. There are a number of pedigree dog registration schemes, of which the Kennel Club is the most well-known.

TABLE 1

| Size Category name | Weight range of dog breed | |
|---|---|---|
| Toy/Extra small | Up to about 6.5 | Kg |
| Small | about 6.5-9 | Kg |
| Medium-small | about 9-15 | Kg |
| Medium-large | about 15-30 | Kg |
| Large | about 30-40 | Kg |
| Giant | Over about 40 | Kg |

In certain embodiments, the dog size categories are selected according to Salt et al., 2017 (Table 1) (Salt et al., 2017. Growth standard charts for monitoring bodyweight in dogs of different sizes PLoS ONE 12: e0182064.). In other embodiments, the dog size categories are selected according to alternative designations. A small breed can correspond with animals that have an average body weight of from about 6.5 kilograms to about 9 kilograms. A medium breed can correspond with an animal that has an average body weight between about 9 kilograms and about 30 kilograms. A large breed can correspond with an animal that has an average body weight of between about 30 kilograms and about 40 kilograms. A giant breed can correspond with an animal that has an average body weight of between over about 40 kilograms.

In other embodiments, the dog size categories are selected according to alternative designations. A small breed can correspond with animals that have an average body weight of up to about 10 kg. A medium breed can correspond with animals that have an average body weight of about 10 kg to about 25 kg. A large breed can correspond with animals that have an average body weight of about 25 kg to about 45 kg. An extra-large breed can correspond with animals that have an average body weight of at least about 45 kg.

It is well known that the daily amount of food to be fed to an animal, can depend on the animal's weight. This is particularly true for dogs. The recommended daily amount of food can be provided as follows in Table 2.

TABLE 2

| Size Category Name | Recommended Daily Amount of Food Per Day |
|---|---|
| Small | about 30 g to about 200 g |
| Medium | about 100 g to about 400 g |
| Large | about 200 g to about 600 g |
| Extra-Large | about 360 g to about 900 g |

In particular embodiments, the amount of glycyrrhizin is from about 5 mg/kg to about 100 mg/kg of bodyweight, from about 10 mg/kg to about 75 mg/kg of bodyweight, from about 15 mg/kg to about 50 mg/kg of bodyweight, or from about 20 mg/kg to about 45 mg/kg of bodyweight based on the total weight of dry matter of the animal food composition.

Consequently, the skilled artisan can easily determine the amount, or amount range, of glycyrrhizin that shall be comprised by weight of a food composition, so that an animal, such as a canine, and especially a dog, will be provided with the preferred daily amount of glycyrrhizin disclosed herein, e.g., as expressed as a daily amount of glycyrrhizin per kg of body weight.

Illustratively, for preventing and/or treating an allergy skin disease, e.g., an atopic dermatitis, in a small dog weighing 9 kg and provided daily with 180 g of a complete food composition, e.g., in the form of kibbles, the amount of glycyrrhizin comprised therein shall be of more than about 0.27 (0.03×9) mg. Thus, the said complete food shall comprise an amount of glycyrrhizin of more than about 0.15 mg per 100 g of complete food, or about 0.015 mg per kg of complete food.

As already described previously herein, the animal food composition comprising glycyrrhizin can comprise a food supplement composition (i.e., an incomplete food composition) or a complete food composition.

### 6.3. Methods of Preparing Animal Food Compositions

The presently disclosed subject matter also encompasses a method for manufacturing the animal food composition specified herein. The process for the manufacture of the animal food composition as described can be made according to any method known in the art.

The animal food composition can be manufactured by mixing together ingredients and kneading in order to make consistent dough or meat emulsion that can be cooked, i.e., a mixture. This applies also to liquids where ingredients are mixed, homogenized before a cooking step in a packaging. The process of creating an embodiment of a dry food is usually done by baking and/or extruding. The dough is typically fed into a machine called an expander and/or extruder, which uses pressurized steam or water to cook the ingredients. While inside the extruder, the dough is under extreme pressure and high temperatures. The dough is then pushed through a die (specifically sized and shaped hole) and then cut off using a knife. The puffed dough pieces are made into kibble by passing it through a dryer so that moisture is dropped down to a defined target ensuring stability of the food until consumption. The kibble can then be sprayed with fats, oils, minerals, vitamins, the natural extracts cocktail, palatants and optionally sealed into packages.

The composition can be a dry food, a wet food or a semi-moist food as described in the presently disclosed subject matter.

Thus, the presently disclosed subject matter provides a method of manufacturing an animal food composition comprising a source of glycyrrhizin wherein it comprises the steps of:
a) preparing a source of glycyrrhizin;
b) optionally mixing one or more additional ingredients and/or additives, thereby providing a mixture; and
c) optionally heating the mixture.

In non-limiting embodiments, the animal food composition manufactured in the method of manufacturing comprises a kibble and/or comprises a source of proteins.

In certain embodiments, the source of glycyrrhizin is mixed in step b), with ingredients selected from the group consisting of a source of curcuminoids, linoleic acid, EPA/DHA, taurine, lutein, vitamin E, and combinations thereof.

In certain embodiments, the source of glycyrrhizin is mixed in step b), with ingredients selected from the group consisting of a source of linoleic acid, EPA/DHA, taurine, lutein, vitamin E, and combinations thereof.

In embodiments, the source of glycyrrhizin is mixed in step b), with ingredients selected from the group consisting of a source of linoleic acid, EPA/DHA, taurine, lutein, vitamin E, protein, crude fat, fibers, or combinations thereof.

### 6.4 Therapeutic Applications

The presently disclosed subject matter provides the use of an animal food composition as described herein for preventing and/or treating inflammatory diseases or disorders, allergic inflammatory skin diseases, preferably for preventing and/or treating atopic dermatitis, including atopic dermatitis in canine animals, especially in dogs.

As discussed herein, allergic inflammatory skin diseases can include at least one of atopic dermatitis, flea allergic dermatitis, urticaria, insect bite allergy, angioedema, inhalant allergy, inhalant allergic dermatitis, food allergic dermatitis, contact dermatitis, miliary dermatitis, eosinophilic granuloma, head and neck pruritus and generalized pruritus, preferably atopic dermatitis, including atopic dermatitis in canine animals, especially in dogs.

In certain embodiments, the animal food composition is used in a method for preventing and/or treating an animal affected with an inflammatory diseases or disorders, an allergic inflammatory skin disease, more particularly of an animal affected with an atopic dermatitis, i.e., a canine atopic dermatitis.

In particular embodiments, the animal food composition is used for preventing and/or treating atopic dermatitis. According to a further embodiment, the animal food composition is used for preventing and/or treating canine atopic dermatitis. According to a yet further embodiment, the animal food composition is used for reducing the dose and frequency of drugs required to manage the condition of inflammatory diseases or disorders, or an allergic inflammatory skin disease.

The presently disclosed subject matter also includes a method for preventing and/or treating inflammatory diseases or disorders, allergic inflammatory skin diseases in an animal comprising at least a step of feeding the animal with an animal food composition according to the presently disclosed subject matter.

In certain embodiments, the animal is affected with an allergic inflammatory skin disease, preferably the animal is affected with an atopic dermatitis, more preferably the animal is a dog affected with a canine atopic dermatitis.

An animal food composition as described herein allows for preventing or treating inflammatory diseases or disorders, allergic inflammatory skin diseases, preferably atopic dermatitis. In certain embodiments, animals are fed exclusively with the food composition described herein. In certain embodiments, animals are fed partly with the composition food described herein and another food composition In certain embodiments, animals are fed with the said food composition as a supplement food or a drug. In any of these disclosed regiments, beneficial effects are obtained.

In certain embodiments, “partly” means that the said animal food composition represents at least about 30% of the animal meal, preferably at least about 30% of said animal meal, or even at least about 70% of said animal meal, or even at least about 80% of said animal meal, or even at least about 90% of said animal meal.

Generally, a foodstuff is provided to animals, especially to dogs, on a daily basis. In certain embodiments, an animal food composition as disclosed herein can be provided to the animal to be treated as the sole nutritionally complete food during the time period of treatment. According to these embodiments, the said nutritionally complete animal food composition is provided to the animal on a daily basis during the time period of treatment.

In certain embodiments, an animal food composition as described herein can be provided to the animal to be treated in alternation with another animal food composition, preferably with another nutritionally complete animal food composition, which other nutritionally complete food composition can be selected among known animal food compositions, including the large variety of commercialized animal food compositions, especially the large variety of commercialized dog food compositions.

In particular embodiments, the said animal food composition is provided each alternate day, thus according to a time schedule of every two days. In certain embodiments, the said animal food composition is provided according to a time schedule of every three, four, five, six, seven, or more days.

It shall be understood that, in the daily practice of feeding animals, especially dogs, the animal owner cannot always feed the animal the animal food composition on a daily basis. In certain embodiments, the beneficial effects of preventing or treating inflammatory diseases or disorders, allergic inflammatory skin diseases, and especially of preventing or treating canine allergic dermatitis, is fully provided when the animal is fed with the animal food composition described herein every alternate days. In particular embodiments, feeding the animal every three, four, five, six or seven days will cause a reduced beneficial effect, which can require a longer time period of treatment.

Without wishing to be bound by any particular theory, in particular embodiments, the efficient prevention or treatment of inflammatory diseases or disorders, allergic inflammatory skin diseases, such as a fully efficient prevention or treatment of canine allergic dermatitis requires that the animal, especially the dog, is provided with an animal food composition as described herein at least on an alternate day basis, most preferably on a daily basis.

The time period of feeding animal food composition as described herein can range from several weeks to several years, depending notably on the severity of the allergic inflammatory skin diseases, i.e., atopic dermatitis.

As already specified in the present disclosure, a short time period, i.e., 1 month, of treatment can be sufficient to decrease pruritus.

In certain embodiments, the animal food composition is provided to the animals to be treated during a longer period of time, such as during a period of time of 9 months or more; such as of 12 months or more, either (i) according to a feeding schedule comprising providing to the animal, especially a dog, exclusively the animal food composition described herein or (ii) according to a schedule alternating the animal food composition described herein and another animal food composition.

The present disclosure also relates to a method for preventing and/or treating inflammatory diseases or disorders, allergic inflammatory skin diseases in an animal affected with allergic inflammatory skin disease comprising at least a step of making a feeding recommendation that includes an animal food composition as defined in the presently disclosed subject matter.

The present disclosure further relates to the use of a source of glycyrrhizin for the preparation of a composition for the treatment of allergic inflammatory skin diseases.

In certain embodiments, the animal food composition inhibits the release of one or more interleukin targets from activated T-cells. As provided herein, overexpression of interleukin-4 (IL-4) and/or interleukin-5 (IL-5) can be indicative of a predisposition of the immune response towards the allergic phenotype. In certain embodiments, the animal food composition inhibits the release of IL-5 from activated T-cells. In certain embodiments, the animal food composition inhibits the release of IL-4 from activated T-cells. In particular embodiments, the animal food composition inhibits the release of IL-5 and IL-4 from activated T-cells. In alternative embodiments, the animal food composition does not stimulate proliferation or release of interleukin-2 (IL-2).

In certain embodiments, the animal food composition can be used for inhibiting release of I-L4 or IL-5 from activated T-cells. In certain embodiments, the animal food composition can be used for preventing and/or treating a disease characterized by increased IL-4 or IL-5 release by activated T-cells. In particular embodiments, the disease characterized by increased IL-4 or IL-5 release by activated T-cells is selected from the group consisting of allergic inflammatory skin diseases, in particular is selected from the group consisting of atopic dermatitis, flea allergic dermatitis, urticaria, insect bite allergy, angioedema, inhalant allergy, inhalant allergic dermatitis, food allergic dermatitis, contact dermatitis, miliary dermatitis, eosinophilic granuloma, head and neck pruritus and generalized pruritus. In particular embodiments, the disease characterized by increased IL-4 or IL-5 release by activated T-cells is atopic dermatitis.

The presently disclosed subject matter also includes a kit comprising a source of glycyrrhizin for use in a method for preventing and/or treating inflammatory diseases or disorders, allergic inflammatory skin diseases, preferably atopic dermatitis, i.e., canine atopic dermatitis.

The present disclosure further relates to the use of a kit comprising a first part comprising a source of glycyrrhizin, and a second part comprising one or more additional ingredients, for the preparation of a composition for the treatment of allergic inflammatory skin diseases.

In certain embodiments, the presently disclosed subject matter relates to a kit comprising a source of glycyrrhizin for use in a method for preventing and/or treating inflammatory diseases or disorders, allergic inflammatory skin diseases wherein the said kit is formulated for providing to an animal a daily amount of glycyrrhizin in an amount of at least about 0.02 mg/kg of bodyweight.

In certain embodiments, the kit as described herein can be provided to the animal to be treated in alternation with another animal food composition or kit, preferably with another nutritionally complete animal food composition or kit, which other nutritionally complete food composition can be selected among known animal food compositions, including the large variety of commercialized animal food compositions, especially the large variety of commercialized dog food compositions.

In particular embodiments, the said kit is provided each alternate day, thus according to a time schedule of every two days. In certain embodiments, the said kit is provided according to a time schedule of every three, four, five, six or seven days.

A kit, as described herein, is used in methods for preventing or treating inflammatory diseases or disorders, allergic inflammatory skin diseases, preferably atopic dermatitis. In certain embodiments, animals fed exclusively with the kit described herein. In certain embodiments, animals are fed partly with the kit described herein and another food composition or kit. In certain embodiments, animals are fed with the said kit as a supplement food or a drug. In any of these disclosed regiments, beneficial effects are obtained.

### 7. EXAMPLES

For the purpose of understanding and not limitation, the presently disclosed subject matter will be better understood by reference to the following examples, which are provided as exemplary of the presently disclosed subject matter, and not by way of limitation.

### Example 1: Glycyrrhizin Safety Study

### Materials and Methods

The purpose of this clinical trial was to demonstrate the safety of three doses of Glycyrrhizin (Gly) in the form of Licorice root extract fed to dogs for 28 days which are much higher that the recommended doses for animal consumption, in particular dog consumption.

Dogs. Sixteen privately owned healthy adult dogs, both male and female, were included in the study. In particular, the dogs were Beagles, Brittany spaniels or Dachshunds weighing between 5 and 21 kg. Twelve dogs were females and four dogs were males. A greater number of females were selected for the study as previous studies in other species showed a greater susceptibility to blood pressure increase upon exposure to Glycyrrhizin.

Glycyrrhizin Source. The glycyrrhizin source was provided by a licorice root extract comprising 12% glycyrrhizin formulated in gelatin capsules. The licorice root extract was commercially obtained from Naturex (Avignon, France), product reference DAB40178. The product was supplied with a certificate of analysis and was shown to contain 12.6% Glycyrrhizin w/w by HPLC method.

Commercially available pharmaceutical grade gelatin capsules (Capsuline, Dania Beach, Florida, US) were filled to capacity with precisely weight mixtures of licorice powder and 100% pure maltodextrin powder (Bulk Powders, Colchester, UK). The weighed combinations of licorice and maltodextrin powders were prepared in such a way that, when mixed together and placed in the capsules, provided the dogs with daily glycyrrhizin doses according to the schedule of 0.2 mg/kg Bwt/day, followed by 0.4 mg/kg Bwt/day, followed by 0.6 mg/kg Bwt/day.

Dosage. The doses to be administered to each dog were calculated based on five different bodyweight groups. Separate capsules were weighed out for each group. The licorice root extract was mixed with maltodextrin order to deliver the same volume of overall powdered material to each dog. Capsules were fed to the dogs inside a meatball of wet petfood.

In total, three different doses of glycyrrhizin were fed to the dogs, in each case for 28 days. These dosages were 0.2 mg/kg of Bwt/day, 0.4 mg/kg of Bwt/day, and 0.6 mg/kg of Bwt/day.

Each of the dogs underwent the following protocol cycle, which was repeated for each of the tested concentrations. The first cycle was dosed at 0.2 mg of glycyrrhizin (Gly)/kg Bwt/day. The second cycle was dosed at 0.4 mg Gly/kg Bwt/day. The third cycle was dosed at 0.6 mg Gly/kg Bwt/day.

### Protocol Cycle:

Day 1: Baseline measurements were taken: blood pressure and potassium, sodium and calcium, creatinine and aldosterone concentrations in the blood and urine.

Day 0: Dosing of licorice began (the concentration of glycyrrhizin is adapted according to the first, second or third cycle of the protocol, as indicated above). One dose was provided to each dog per day, with their morning meal.

Days 1-27: One dose was provided to each dog per day, with their morning meal.

Days 2, 4, 7, 14, 21 and 28: Blood pressure and potassium, sodium and calcium, creatinine and aldosterone concentrations in the blood and urine measurements were taken.

Day 28: Washout began and the dogs returned to their maintenance diet alone, i.e., no more licorice was fed to them. The washout period lasted two weeks. At days 34 and 41 blood pressure and potassium, sodium and calcium, creatinine and aldosterone concentrations in the blood and urine measurements were taken again.

If there was a significant change in the dog's measurements at the end of the cycle compared to the baseline measurements, the study ended. If there was not a significant change in the dog's measurements at the end of the cycle compared to the baseline measurements, the dog continued on to the next cycle of the protocol.

Protocol Details. The licorice root extract was dosed according to the given glycyrrhizin (Gly) concentrations for each cycle of the protocol in sequence: 0.2 mg/kg Bwt/day, 0.4 mg/kg Bwt/day, and 0.6 mg/kg Bwt/day. The second and third cycles of the protocol was initiated only after it has been shown that the previous cycle did not result in a significant change in blood pressure (BP) or blood and urine parameters.

Blood pressure was measured for each animal on multiple occasions, the first reading being discarded. BP was measured via the Oscillometric method. Each measurement, if it was shown that a dog's average blood pressure has risen to a potentially deleterious level (normally >160 mmgHg), could result in removal of a dog from the study (temporarily or permanently), according to veterinarian's discretion. All dogs were habituated to the Oscillometic device and the process of BP measurement before the start of the study. If there was evidence of stress for any dog before or during the BP procedure, the procedure for that dog was stopped and restarted when the dog was calmed again. Where necessary a dog could be given interim intensive habituation. If these measures failed to address the problem, the dog was permanently removed from the study and replaced by a closely matched alternative animal. Typical blood pressure ranges for the 3 breeds used were known and were considered when judging the normality of the BP measurements taken during the study. For the three breeds used the normal reference ranges for BP were:
Beagle: Systolic 140+/−15, Diastolic 79+/−13
Dachshund: Systolic 142+/−10, Diastolic 85+/−15
Brittany Spaniel: Systolic 131+/−16, 74+/−14

The conditions under which the BP measurements were taken in accordance with the following procedure.

The environment was isolated and quiet. The patient was sedated and was allowed to acclimate to the measurement room for 5-10 minutes before the measurement was attempted. The animal was then gently restrained in a comfortable position, ideally in ventral or lateral recumbency to limit the vertical distance from the heart base to the cuff.

The cuff width was approximately 30%-40% of circumference of the cuff site. The cuff size was tracked, and the same size was used each time. The cuff was placed on the right front leg which was pre-clipped where the probe was applied. This resulted in less stress and time pressure on the day of collections. The right front leg was used on all animals for each measurement. Wherever possible the same individual performed blood pressure measurements on each occasion for the same dog.

The measurements were taken only when the patient was calm and motionless. The first measurement was discarded. A total of 5 consecutive consistent values were recorded. In some patients, measured blood pressure trended downward as the measurements were taken. In these animals, measurements were continued until the decline levelled off and then 5 consecutive consistent values were recorded.

Blood pressure measurements were repeated as necessary, changing cuff placement as needed to obtain consistent values. Blood pressure values were based on a mean of the 5 measurements recorded.

Small volume blood samples (4 ml) were taken on the days indicated in the protocol description above in order to determine blood potassium, sodium and calcium, creatinine and aldosterone concentration. Free catch urine (minimum 3 ml) was conducted on each day as indicated above in order to determine concentrations of urinary potassium, sodium and calcium. Urinary creatinine concentrations were also measured so that Fractional Excretion Rate (FER) for sodium, potassium and calcium could be calculated.

FER is calculated according to the equation:

$$( Urinary Potassium \times Serum Creatinine \times 100 ) / Serum Potassium \times Urinary Creatinine ) .$$

As licorice root extract (LRE)/Gly was evaluated as a single ingredient, it was most effective to dose directly using measured quantities of licorice in capsules. Therefore, the appropriate dose of LRE was weighed into gelatin capsules and then dosed alongside the normal feeding schedule of the dogs.

Statistics: A linear mixed model was used for assessing the effect of Diet (Glycyrrhizin dose). Animal was modelled as random term. Tukey HSD was used for alpha correction for multiple comparisons. Level of significance was set at 5% for two-sided test.

Two ways of modelling blood pressure were assessed. In the first method, blood pressure difference between baseline and after 4 weeks was calculated for each dog and each sequence and set as dependent variables. In the second method, blood pressure after 4 weeks was used as dependent variable and blood pressure at baseline as covariable of the model.

### Results

Blood Pressure. The overall mean systolic blood pressure (mmHg) for each tested dose of glycyrrhizin (Gly) over time is provided in Table 3 below:

TABLE 3

| | | | | | Day | Day | Day |
|---|---|---|---|---|---|---|---|
| Means | Day 0 | Day 2 | Day 4 | Day 7 | 14 | 21 | 28 |
| 0.2 mg Gly | 120 | 127 | 125 | 126 | 126 | 122 | 121 |
| 0.4 mg Gly | 118 | 117 | 119 | 125 | 122 | 120 | 124 |
| 0.6 mg Gly | 125 | 119 | 123 | 125 | 123 | 124 | 122 |

These results show that there was no significant difference in blood pressure between DO and any later time point for any of the tested doses.

Fractional Potassium Excretion. The overall mean fractional potassium excretion for each tested dose of glycyrrhizin (Gly) over time is provided in Table 4 below. The results are expressed in percentage of the potassium excreted in the urine as compared to the amount of potassium filtered and retained by the kidneys.

TABLE 4

| | | | | | Day | Day | Day |
|---|---|---|---|---|---|---|---|
| Means | Day 0 | Day 2 | Day 4 | Day 7 | 14 | 21 | 28 |
| 0.2 mg Gly | 16.54 | 15.74 | 15.59 | 15.06 | 13.33 | 20.02 | 16.54 |
| 0.4 mg Gly | 16.02 | 15.56 | 16.02 | 15.72 | 16.87 | 16.09 | 16.27 |
| 0.6 mg Gly | 16.48 | 18.08 | 17.48 | 13.15 | 15.64 | 14.93 | 16.48 |

These results show that there was no significant difference in fractional potassium excretion between DO and any later time point at any of the tested doses.

Plasma Aldosterone Concentration. The overall mean Plasma Aldosterone concentration in pmol/L, for each tested dose of glycyrrhizin (Gly) over time is provided in 15 Table 5 below:

TABLE 5

| Means | Day 0 | Day 7 | Day 14 | Day 28 |
|---|---|---|---|---|
| 0.2 mg Gly | 62.13 | 45.13 | 49.75 | 44.83 |
| 0.4 mg Gly | 54.00 | 52.25 | 56.38 | 70.57 |
| 0.6 mg Gly | 36.50 | 42.57 | 59.00 | 63.25 |

These results show that there was no significant difference in plasma aldosterone concentration between DO and any later time point at any of the tested doses.

In conclusion, based on the results of this study, there was no significant impact of Glycyrrhizin on blood pressure homeostasis, fractional potassium excretion and plasma aldosterone concentration in adult dogs for the doses and duration tested.

### Example 2: Immunomodulatory Potential of Glycyrrhizin

### Materials and Methods

Licorice root extract preparation for bioassays. A licorice root extract (LRE) was obtained from William Ransom & Son PLC (UK). For preparation, 20 g of the root powder was suspended in 100 ml of a solution of 20% ethanol in distilled water. The suspension was then heated with moderate agitation to 60° C. for 2 hours and then the resulting extract filtered through a Whatman No. 4 filter paper. This extraction process was repeated once more with the filtered solid material. The pooled filtrate was then concentrated in a rotary evaporator at 40° C. overnight to create an extract in the form of a paste. Fresh phytochemical extract was stored at −20° C. under nitrogen. According to supplier's specification the glycyrrhizin concentration of the extract was 18% w/w. Concentrations of LRE are based on the wet weight of the extract. All assays were performed with the same extract.

The Licorice root extract was weighed and suspended at 100 mg/mL in dimethyl sulphoxide (DMSO—Sigma, UK) and further diluted to 4 mg/mL in complete tissue culture medium (CTCM; RPMI-1640 medium (Sigma) plus foetal bovine serum (Sigma) to 5%, 2 mM L-glutamine (Sigma), 100 U/mL penicillin and 100 μg/mL streptomycin (Sigma). These stocks were further diluted in CTCM to deliver concentrations between 0.3 and 1000 μg/mL final concentrations in the assay wells.

Isolation of human peripheral blood mononuclear cells (HuPBMC). HuPBMC were isolated using the method of Telford et al. (Telford et al., International Immunopharmacology. 4 (2004) 1455-1466). Briefly, heparinised blood was obtained with consent from healthy human volunteers aged 20-50 years (Nottingham University Medical School Research Ethics Committee, BT/04/2005), diluted in an equal volume of RPMI-1640 medium (Sigma) and layered onto Histopaque 1077 (Sigma). After centrifugation at 710×g for 20 minutes, the buffy layer was harvested and washed twice with RPMI-1640. Cells were suspended at 106/mL in CTCM. Mononuclear cell concentration was 85%, as determined by cytology.

LRE cytotoxicity against Jurkat E6.1 T-cell line and HuPBMC. Cytotoxicity was assessed using the human T-lymphocytic leukemia Jurkat E6.1 cell line (Walsh et al., 2008). Pre-diluted LRE extract was tested in triplicate at final concentrations ranging from 0.3 to 1000 μg/mL in 200 μL of CTCM, containing 10⁵ Jurkat E6.1 cells per well in 96-well U-bottomed plates (Costar). After 24 h incubation at 37° C. in 5% CO₂, cell viability was assessed using dye exclusion (20 μL 0.4% Trypan blue in saline (Sigma, UK)) counting 10 random fields of an object lens graticule under a microscope. Percentage survival determined by comparison to a control incubation of cells without LRE.

A similar cytotoxicity assay to the above was performed using normal, fresh human peripheral blood mononuclear cells. The same pre-diluted extracts were tested in triplicate over a 0.1-1000 μg/ml) range, incubated for 24 Hours with 10⁵ HuPBMC at 37° C. in 5% CO₂. Cell viability was assessed as previously described using Trypan blue dye exclusion.

Percentage survival was determined by comparison with cells incubated without LRE. Proliferation and cytokine release assays for HuPBMC. Anti-CD3 antibody (BD Pharmingen, UK) combined with anti-CD28 antibody (BD Pharmingen, UK) was used to stimulate the HuPBMC T-cell proliferation and cytokine expression to assess the modulatory effect of licorice extract. Cell proliferation was determined by [3H]-thymidine incorporation. All in vitro assays were performed with 6 assay replicates.

Pre-diluted root extract (LRE) was tested at final concentrations ranging from 0.3 to 1000 μg/mL in 200 μL of CTCM, containing 10⁵ HuPBMC per well in 96-well U-bottomed plates. The assay used sub-optimal levels of anti-CD3 and anti-CD28 (100 ng/mL final assay concentration of each). The use of sub-optimal stimulation allows determination of potential stimulatory as well as suppressive effects. Dexamethasone alone was also tested in triplicate at a final concentration of 0.5 μM as a positive control (maximal immune suppression). DMSO alone was tested as a vehicle control.

For the Interleukin-2 assay 50 μL culture medium was removed from each well after 24 h incubation at 37° C. in 5% CO₂ and stored at −20° C. until analyzed for IL-2 concentration. At this time the cells were pulsed with 0.25 μCi per well [3H] thymidine (GE Healthcare, UK) and incubated for a further 24 h prior to harvesting onto GF/c filter plates (Packard, UK) using a Packard Filtermate harvester. The radioactivity retained on filters was determined using a Packard Topcount NXT scintillation counter and EC₅₀ (effective concentration reducing proliferation by 50%) determined by non-linear regression using Prism 3.0 software. For the remaining cytokine assays, 150 μL culture medium was removed from each well (immediately prior to cell harvesting) after incubation for 48 h at 37° C. in 5% CO₂ and stored at −20° C. prior to IL-2, IL-5, IL-17 and IFN-7 analysis.

Cytokine Analyses. Concentrations of IL-2, IL-5, IL-17 and IFN-γ in the conditioned culture medium were determined by ELISA according to the following protocol: ninety-six well assay plates (Nunc, UK) were coated with 50 μL of the relevant capture antibody (BD Pharmingen, UK; αIL-2, 555051; αIL-5, 554393; αIFN-γ, 551221; αIL-17, 560486) at 1 μg/mL in carbonate/bicarbonate buffer pH 9.6 and incubated overnight at 4° C. Wells were then incubated with 200 μL 1% bovine serum albumin (BSA, Sigma, UK) in phosphate buffered saline (PBS) for 1 h at room temperature, prior to washing three times with PBS+0.05% tween 20 (Sigma, UK). 50 μL cell culture supernatant samples were then diluted in an equal volume of PBS/tween+1% BSA. Prepared samples and cytokine standards (Human, BD Pharmingen, UK: IL-2, 555602; IL-5, 554606; IFN-γ, 554616. IL-17 Sigma, H7791) were added to relevant wells. After incubation overnight at 4° C., wells were washed three times with PBS/tween and incubated at room temperature for 1 h with 50 μL of the relevant biotinylated detection antibody (Human, BD Pharmingen, UK: αIL-2, 555040; αIL-5, 554491; αIFN-γ, 554550; IL-17, 555067) at 0.5 g/mL in PBS/tween+1% BSA added per well. Washed plates were incubated with 50 μL/well 1:1000 streptavidin/peroxidase (BD Pharmingen, UK) in PBS/tween+1% BSA for 30 minutes at room temperature and, after a final wash with PBS/tween, incubated with 100 μL per well 0.1 mg/mL tetramethylbenzidine substrate (Sigma, UK) in 0.1 M acetate buffer pH 6. The reaction was stopped by the addition of 20 μL 2M H2SO4 and plates read at 450 nm on a Dynex MRX plate reader.

Statistical Analysis. For cytokine assays LRE concentrations were compared to anti-CD3/CD28 stimulus-only control. A one-way analysis of variance followed by a Dunnett's post-test was used to determine significant suppression of cell survival, proliferation and cytokine secretion using JMP14. For the Dunnett post hoc comparison the reference data was provided by the stimulation only positive control incubation.

### Results

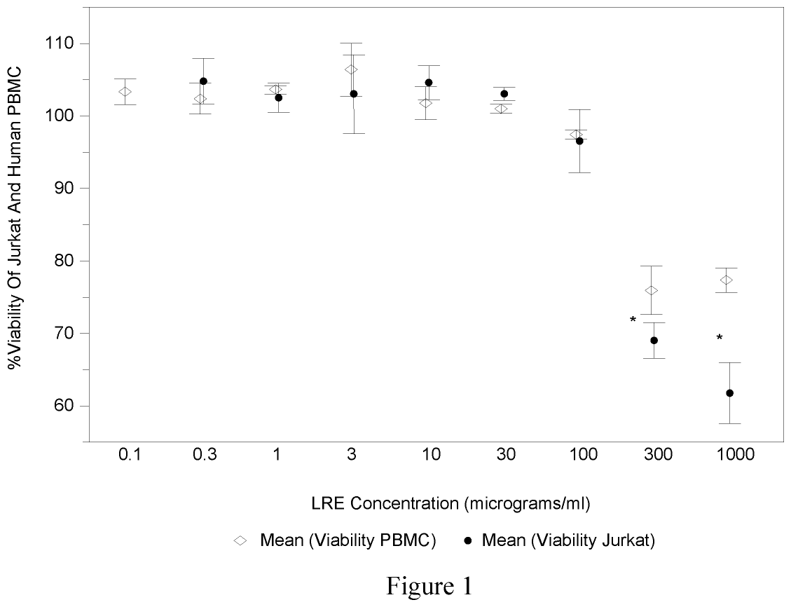

Cytotoxicity of Licorice Root Extract Against Jurkat E6.1 T-Cell Line and Human PBMC. Jurkat E6.1 cells have previously been used to assess the potential immunotoxicity of compounds (Walsh et al., Marine Drugs. 6 (2008) 291-307). Here it provides a useful method to determine whether LRE could induce cell stress/death to mononuclear cells within the concentration range used in subsequent immunomodulation assays. The licorice root extract showed no evidence of toxicity against the Jurkat cell line at concentrations up to 100 μg/ml (FIG. 1). At 300 μg/ml there was evidence for cytotoxicity, with approximately 30% cell death (p<0.01), increasing to 40% at 1 mg/ml extract (p<0.01). It was concluded that a maximum safe concentration of 100 μg/ml extract was acceptable for interpretation of immunological cell assays.

A similar toxicity assay performed using the HuPBMC cells gave comparable results (FIG. 1). Cell viability again decreased at an extract concentration of 300 μg/ml (p<0.01) and although this did not decrease further at 1 mg/ml, the outcome further confirmed that the maximum interpretable extract concentration for cell assays was 100 μg/ml.

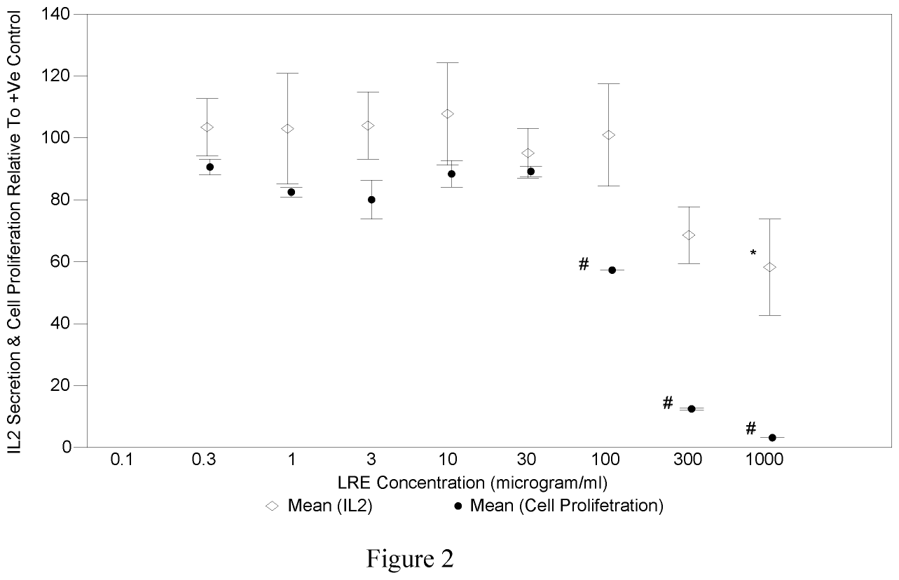

Anti-CD3 CD28 stimulated proliferation and interleukin-2 release by HuPBMC. Proliferation of the HuPBMC population was determined in the presence of the LRE. Alongside CD3/CD28 stimulation, the impact of the LRE was assessed across the 0.3-1000 μg/ml range (FIG. 2). In the range of 0.3-30 μg/ml LRE there was no evidence of an impact. Proliferation was reduced approximately 40% reduction at 100 μg/ml. Proliferation is therefore affected by the LRE at a concentration below the cytotoxic threshold.

Interleukin-2 (IL-2) is a critical cytokine produced by activated T-cells, in that it has a role in controlling immune cell development as well as proliferation. As such, it can be employed as an indicator of the activated state of a population of mononuclear cells. When assessed in the T-cell activation model there was no evidence that interleukin-2 release was influenced by the LRE over the concentration range of 0.3-100 μg/ml (FIG. 2). There was an approximate 30% decrease in secretion of IL-2 at 300 μg/ml LRE however, and a decrease by a further 10% at 1000 μg/ml LRE.

To summarize these observations, there was evidence of a cytotoxic impact of the LRE at concentrations of 300 μg/ml and above. However, the proliferative response was more sensitive to the extract with a concentration of 100 μg/ml causing a significant reduction (p<0.05). Interleukin-2 secretion did not seem to be as sensitive to the LRE as cell proliferation, with 300 μg/ml being the first concentration to elicit a significant reduction (p<0.05). The impact at higher concentrations was not so dramatic, however.

Two possible interpretations of the observations at high LRE concentrations were that the anti-proliferative and anti-IL-2 effects are simply linked to cell stress/death and could be indicators of incipient cells stress, detectable prior to the onset of cell death. Alternatively, the anti-proliferation and anti-IL-2 effects could be distinct from cell death.

Interleukin-5, Interleukin-17 and Interferon-γ expression by stimulated HuPBMC. In order to elucidate further the potential immunomodulatory actions of the LRE, and in particular to determine its impact on different T-helper (Th) cell types, titrated concentrations of the extract were incubated with anti-CD3/CD28 activated HuPBMC. Th1 cell activity was inferred by quantifying Interferon-γ secretion in the culture medium.

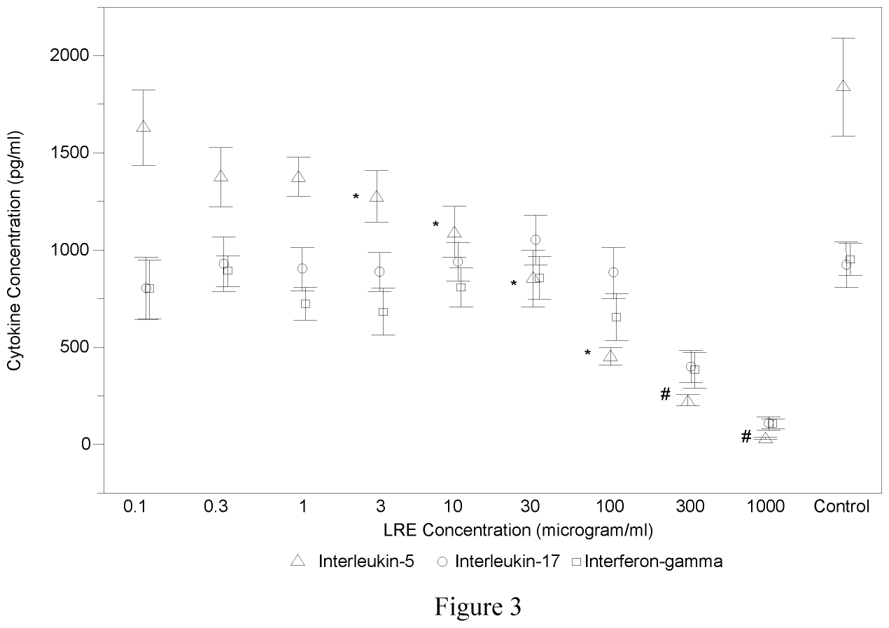

Similarly, Th2 activity was detected by quantifying the proxy Interleukin-5. Finally, Th17 activity was inferred by detection of interleukin-17 (IL-17) (FIG. 3). Immunomodulatory effects were assessed by comparing cytokine secretion relative to that observed in the presence of activating agents alone (anti-CD3/CD28 for human), thereby calibrating to 100% activity.

In the HuPBMC assays, there was no significant reduction in secretion of interferon-γ (IFNγ) nor IL-17 between LRE concentrations of 0.1-10 μg/ml. Interleukin-5 secretion however was seen to be significantly decreased at 3 and 10 μg/ml, showing 30% (P<0.05) and 40% (p<0.005) reductions respectively relative to the positive control. The reduction was greater at 30 μg/ml LRE concentration, showing >50% reduction on the positive control (P<0.0001), at 100 μg/ml, showing a 75%% reduction (P<0.0001). At LRE concentrations above 100 μg/ml expression of all 3 cytokines was suppressed significantly relative to the positive control (P<0.0001). At these higher concentrations the observed effects were probably associated with cytotoxicity of the LRE. Differential effects on cytokines were therefore interpreted at concentrations below 100 μg/ml. The 100 μg/ml concentration was a little more ambiguous due to the observed impact on proliferation. At concentrations above 100 μg/ml cytotoxicity was likely to explain all observations. A summary of the differential effects of LRE on the three cytokines is shown in the results presented in the figures that are referred to above.

### CONCLUSION

In summary, the inventors have provided evidence that an aqueous extract of licorice root is able to inhibit release of IL-5 from activated T-cells within a concentration range which is non-cytotoxic and does not stimulate proliferation nor IL-2 release. What is more the concentration range does not significantly impact the Th-1 cytokine IFN-7 nor the Th-17 cytokine IL-17.

Although the presently disclosed subject matter and its advantages have been described in detail, it should be understood that various changes, substitutions and alterations can be made herein without departing from the spirit and scope of the disclosed subject matter. Moreover, the scope of the present application is not intended to be limited to the particular embodiments of the process, and composition of matter, methods and processes described in the specification. As one of ordinary skill in the art will readily appreciate from the disclosed subject matter of the presently disclosed subject matter, processes, compositions of matter, methods, or steps, presently existing or later to be developed that perform substantially the same function or achieve substantially the same result as the corresponding embodiments described herein may be utilized according to the presently disclosed subject matter. Accordingly, the appended claims are intended to include within their scope such processes, machines, manufacture, compositions of matter, methods, or steps.

Various patents, patent applications, publications, product descriptions, protocols, and sequence accession numbers are cited throughout this application, the inventions of which are incorporated herein by reference in their entireties for all purposes.

1. A method for preventing and/or treating inflammatory diseases or disorders, or allergic inflammatory skin diseases in an animal in need thereof, the method comprising providing an animal food composition, wherein the animal food composition comprises glycyrrhizin in an amount of at least about 0.02 mg/kg of bodyweight, and wherein the animal food composition is provided daily.

2. The method of claim 1, wherein the animal food composition is formulated for providing to an animal a daily amount of glycyrrhizin in an amount of from about 0.02 mg/kg of bodyweight to about 2 mg/kg of bodyweight.

3. The method of claim 1, wherein the said animal food composition is formulated for providing to an animal a daily amount of glycyrrhizin in an amount of from about 0.03 mg/kg to about 2 mg/kg of bodyweight.

4. The method of claim 1, wherein the animal food composition is selected from the group consisting of a nutritionally complete animal food composition, a food supplement, and a topper composition.

5. The method of claim 1, wherein a source of the glycyrrhizin comprises a licorice root extract.

6. The method of claim 1, wherein the animal food composition further comprises linoleic acid.

7. The method of claim 1, wherein the animal food composition further comprises EPA/DHA, Taurine, Lutein, Vitamin E or a combination thereof.

8. The method of claim 1, wherein said-allergic inflammatory skin disease is selected from the group consisting of atopic dermatitis, flea allergic dermatitis, urticaria, insect bite allergy, angioedema, inhalant allergy, inhalant allergic dermatitis, food allergic dermatitis, contact dermatitis, miliary dermatitis, eosinophilic granuloma, head and neck pruritus, and generalized pruritus.

9. A kit comprising:
(i) a first part comprising a source of glycyrrhizin; and
(ii) a second part comprising one or more additional ingredients;
for use in a method for preventing and/or treating allergic inflammatory skin diseases wherein the said kit is formulated for providing to an animal a daily amount of glycyrrhizin in an amount of at least about 0.02 mg/kg of body weight.

10. An animal food composition comprising a source of glycyrrhizin, wherein the amount of glycyrrhizin is at least about 5 mg/kg based on the total weight of dry matter of the animal food composition.

11. The animal food composition of claim 10, wherein the amount of glycyrrhizin is from about 5 mg/kg to about 100 mg/kg based on the total weight of dry matter of the animal food composition.

12. The animal food composition of claim 10, wherein the amount of glycyrrhizin is from about 10 mg/kg to 75 mg/kg based on the total weight of dry matter of the animal food composition.

13. The animal food composition of claim 10, wherein the animal food composition comprises a kibble.

14. The animal food composition of claim 10, wherein the animal food composition comprises a nutritionally complete food composition.

15. The animal food composition of claim 10, wherein the animal food composition is formulated for oral administration.

16. The animal food composition of claim 10, wherein the source of glycyrrhizin consists of a licorice root extract.

17. The animal food composition of claim 10, further comprising one or more additional ingredients.

18. The animal food composition of claim 17 wherein the one or more additional ingredients are selected from the group consisting of linoleic acid, EPA/DHA, Taurine, Lutein, Vitamin E and a combination thereof.

19. The animal food composition according to claim 10, wherein the animal food composition further comprises a source of proteins.
